(19) United States
(12) Patent Application Publication
(10) Pub. No.: US 2005/0020644 A1
(43) Pub. Date: Jan. 27, 2005

(54) Bicyclic fused pyridinyl amides and advantagesous compositons thereof for use as fungicides

(76) Inventors: John Joseph Bisaha, Hockessin (DE); James Volney Hay, Newark (DE); Stephen Ray Foor, Hockessin (DE); Michael Paul Walker, Landenberg, PA (US); David Alan Clark, Landenberg, PA (US)

Correspondence Address: E I Du Pont de Nemours and Company;Legal-Patents, 4417 Lancaster Pike, Wilmington, DE, 19898, US

(21) Appl. No.: 10/501,258

(22) PCT Filed: Feb. 20, 2003
(86) PCT No.: PCT/US03/05383

Related U.S. Application Data
(60) Provisional application No. 60/365,767, filed on Mar. 19, 2002.

(51) Int. Cl. C07D 213/56; A01N 43/40

(52) U.S. Cl. 514/355; 546/316

(57) ABSTRACT

Compounds of Formula I, including all geometric and stereoisomers, N-oxides, and agriculturally suitable salts thereof: (Formula I); wherein X and either Y or Z are a linking chain 3 or 4 atoms in length attached to contiguous carbon atoms and are taken together with said carbon atoms to form a fused phenyl ring, a fused 5- or 6-membered nonaromatic carbocyclic or heterocyclic ring optionally including one or two ring members selected from the group consisting of C(═O), SO or S(O)₂, or a fused 5- or 6-membered heteroaromatic ring, each fused ring optionally substituted with one to three substituents independently selected from R⁷; and R¹, R², R⁵, R⁶R⁷, m and p are as defined in the disclosure. Also disclosed are compositions containing the compounds of Formula I and a method for controlling plant diseases caused by fungal plant pathogens that involves applying an effective amount of a compound of Formula I.

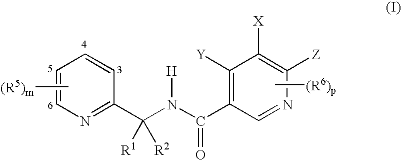

### BACKGROUND OF THE INVENTION

This invention relates to certain bicyclic fused pyridinyl amides, their N-oxides, agriculturally suitable salts and compositions, and methods of their use as fungicides.

The control of plant diseases caused by fungal plant pathogens is extremely important in achieving high crop efficiency. Plant disease damage to ornamental, vegetable, field, cereal, and fruit crops can cause significant reduction in productivity and thereby result in increased costs to the consumer. Many products are commercially available for these purposes, but the need continues for new compounds, which are more effective, less costly, less toxic, environmentally safer or have different modes of action.

WO 01/11966 discloses certain pyridinyl amides of formula i as fungicides 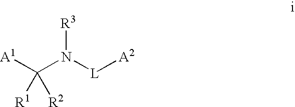
wherein, among others,
A¹ is 2-pyridyl substituted by up to four groups at least one of which is haloalkyl;
A² is optionally substituted heterocyclyl;
R¹ and R² are independently H, alkyl or acyl;
R³ is H or allyl; and
L is —(C═O)—, —SO₂— or —(C═S)—.

Fungicides that effectively control plant fungi, particularly of the class Oomycetes, such as Phytophthora spp. and Plasmopara spp., are in constant demand by growers. Combinations of fungicides are often used to facilitate disease control and to retard resistance development. It is desirable to enhance the activity spectrum and the efficacy of disease control by using mixtures of active ingredients that provide a combination of curative, systemic and preventative control of plant pathogens. Also desirable are combinations that provide greater residual control to allow for extended spray intervals. It is also very desirable to combine fungicidal agents that inhibit different biochemical pathways in the fungal pathogens to retard development of resistance to any one particular plant disease control agent.

It is in all cases particularly advantageous to be able to decrease the quantity of chemical agents released in the environment while ensuring effective protection of crops from diseases caused by plant pathogens. Mixtures of fungicides may provide significantly better disease control than could be predicted based on the activity of the individual components. This synergism has been described as “the cooperative action of two components of a mixture, such that the total effect is greater or more prolonged than the sum of the effects of the two (or more) taken independently” (see Tames, P. M. L., Neth. J. Plant Pathology, (1964), 70, 73-80).

There is a desire to find fungicidal agents that are particularly advantageous in achieving one or more of the preceding objectives.

### SUMMARY OF THE INVENTION

This invention relates to compounds of Formula I (including all geometric and stereoisomers), N-oxides, and agriculturally suitable salts thereof: 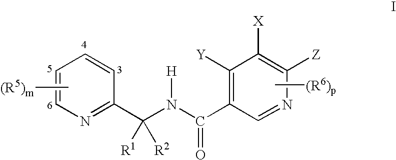
wherein
R¹ and R² are each independently H or C₁-C₆ alkyl;
X and either Y or Z are a linking chain 3 or 4 atoms in length attached to contiguous carbon atoms and are taken together with said carbon atoms to form a fused phenyl ring, a fused 5- or 6-membered nonaromatic carbocyclic or heterocyclic ring optionally including one or two ring members selected from the group consisting of C(═O), SO and S(O)₂, or a fused 5- or 6-membered heteroaromatic ring, each fused ring optionally substituted with one to three substituents independently selected from R⁷;
each R⁵ is independently C₁-C₆ alkyl, C₂-C₆ alkenyl, C₂-C₆ alkynyl, C₃-C₆ cycloalkyl, C₁-C₆ haloalkyl, C₂-C₆ haloalkenyl, C₂-C₆ haloalkynyl, C₃-C₆ halocycloalkyl, halogen, CN, CO₂H, CONH₂, NO₂, hydroxy, C₁-C₄.alkoxy, C₁-C₄ haloalkoxy, C₁-C₄ alkylthio, C₁-C₄ alkylsulfinyl C₁-C₄ alkylsulfonyl, C₁-C₄ haloalkylthio, C₁-C₄ haloalkylsulfinyl, C₁-C₄ haloalkylsulfonyl, C₁-C₄ alkylamino, C₂-C₈ dialkylamino, C₃-C₆ cycloalkylamino, C₂-C₆ alkylcarbonyl, C₂-C₆ alkoxycarbonyl, C₂-C₆ alkylaminocarbonyl, C₃-C₈ dialkylamninocarbonyl or C₃-C₆ trialkylsilyl;
each R⁶ is independently C₁-C₆ alkyl, C₂-C₆ alkenyl, C₂-C₆ alkynyl, C₃-C₆ cycloalkyl, C₁-C₆ haloalkyl, C₂-C₆ haloalkenyl, C₂-C₆ haloalkynyl, C₃-C₆ halocycloalkyl, halogen, CN, CO₂H, CONH₂, NO₂, hydroxy, C₁-C₄ alkoxy, C₁-C₄ haloalkoxy, C₁-C₄ alkylthio, C₁-C₄ alkylsulfinyl, C₁-C₄ alkylsulfonyl, C₁-C₄ haloalkylthio, C₁-C₄ haloalkylsulfinyl, C₁-C₄ haloalkylsulfonyl, C₁-C₄ alkylamino, C₂-C₈ dialkylamino, C₃-C₆ cycloalkylamino, C₂-C₆ alkylcarbonyl, C₂-C₆ alkoxycarbonyl, C₂-C₆ alkylaminocarbonyl, C₃-C₈ dialkylaminocarbonyl or C₃-C₆ trialkylsilyl;
each R⁷ is independently C₁-C₄ alkyl, C₂-C₄ alkenyl, C₂-C₄ alkynyl, C₃-C₆ cycloalkyl, C₁-C₄ haloalkyl, C₂-C₄ haloalkenyl, C₂-C₄ haloalkynyl, C₃-C₆ halocycloalkyl, halogen, CN, NO₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ alkylamino, C₂-C₈ dialkylamino, C₃-C₆ cycloalkylamino, C₃-C₆ (alkyl)cycloalkylamino, C₂-C₄ alkylcarbonyl, C₂-C₆ alkoxycarbonyl, C₂-C₆ alkylaminocarbonyl, C₃-C₈ dialkylaminocarbonyl or C₃-C₆ trialkylsilyl;
m is 1,2, 3 or 4; and
p is 0, 1, or 2.

This invention also relates to fungicidal compositions comprising fungicidally effective amounts of the compounds of the invention and at least one additional component selected from the group consisting of surfactants, solid diluents, liquid diluents and other fungicides.

For example, this invention provides compositions comprising (a) at least one compound of Formula I; and
(b) at least one compound selected from the group consisting of
(b1) alkylenebis(dithiocarbamate)fungicides;
(b2) compounds acting at the bc₁ complex of the fungal mitochondrial respiratory electron transfer site;
(b3) cymoxanil;
(b4) compounds acting at the demethylase enzyme of the sterol biosynthesis pathway;
(b5) morpholine and piperidine compounds that act on the sterol biosynthesis pathway;
(b6) phenylamide fungicides;
(b7) pyrimidinone fungicides;
(b8) phthalimides; and
(b9) fosetyl-aluminum.

This invention also relates to a method for controlling plant diseases caused by fungal plant pathogens comprising applying to the plant or portion thereof, or to the plant seed or seedling, a fungicidally effective amount of a compound or composition of the invention.

### DETAILS OF THE INVENTION

In the above recitations, the term “alkyl”, used either alone or in compound words such as “alkylthio” or “haloalkyl” includes straight-chain or branched alkyl, such as, methyl ethyl, n-propyl, i-propyl, or the different butyl, pentyl or hexyl isomers. “Alkenyl” includes straight chain or branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight chain or branched alkynes such as ethynyl, 1-propynyl, 2-propynyl and the different butynyl, pentynyl and hexynyl isomers. “Alkynyl” can also include moieties comprised of multiple triple bonds such as 2,5-hexadiynyl. “Alkoxy” includes, for example, methoxy, ethoxy, n-propyloxy, isopropyloxy and the different butoxy, pentoxy and hexyloxy isomers. “Alkoxyalkyl” denotes alkoxy substitution on alkyl. Examples of “alkoxyalkyl” include CH₃OCH₂, CH₃OCH₂CH₂, CH₃CH₂OCH₂, CH3CH₂CH₂CH₂OCH₂ and CH₃CH₂OCH₂CH₂. “Alkoxyalkoxy” denotes alkoxy substitution on alkoxy. The term “Alkenyloxy” includes straight chain or branched alkenyloxy moieties. Examples of “alkenyloxy” include H₂C═CHCH₂O, (CH₃)₂C═CHCH₂O, (CH₃)CH═CHCH₂O, (CH₃)CH═C(CH₃)CH₂O and CH₂═CHCH₂CH₂O. “Alkynyloxy” includes straight chain or branched alkynyloxy moieties. Examples of “alkynyloxy” include HC≡CCH₂O, CH₃C≡CCH₂O and CH₃C≡CCH₂CH₂O. “Alkylthio” includes branched or straight chain alkylthio moieties such as methylthio, ethylthio, and the different propylthio, butylthio, pentylthio and hexylthio isomers. “Alkylthioalkyl” denotes alkylthio substitution on alkyl. Examples of “alkylthioalkyl” include CH₃SCH₂, CH₃SCH₂CH₂, CH₃CH₂SCH₂, CH₃CH₂CH₂CH₂SCH₂ and CH₃CH₂SCH₂CH₂. “Alkylthioalkoxy” denotes alkylthio substitution on alkoxy. “Alkylsulfinyl” includes both enantiomers of an alkylsulfinyl group. Examples of “alkylsulfinyl” include CH₃S(O), CH₃CH₂S(O), CH₃CH₂CH₂S(O), (CH₃)₂CHS(O) and the different butylsulfinyl, pentylsulfinyl and hexylsulfinyl isomers. Examples of “alkylsulfonyl” include CH₃S(O)₂, CH₃CH₂S(O)₂, CH₃CH₂CH₂S(O)₂, (CH₃)₂CHS(O)₂ and the different butylsulfonyl, pentylsulfonyl and hexylsulfonyl isomers. “Cyanoalkyl” denotes an alkyl group substituted with one cyano group. Examples of “cyanoalkyr” include NCCH₂, NCCH₂CH₂ and CH₃CH(CN)CH₂. “Alkylamino”, “dialkylamino”, “alkenylthio”, “alkylsulfinyl”, “alkylsulfonyl”, “alkynylthio”, “alkynylsulfinyl”, “alkynylsulfonyl”, and the like, are defined analogously to the above examples. “Cycloalcyl” includes, for example, cyclopropyl, cyclobutyl, cyclopentyl, and cyclohexyl. The term “cycloalkoxy” includes the same groups linked through an oxygen atom such as cyclopentyloxy and cyclohexyloxy.

The term “halogen”, either alone or in compound words such as “haloalkyl”, includes fluorine, chlorine, bromine or iodine. Further, when used in compound words such as “haloalkyl”, said alkyl may be partially or fully substituted with halogen atoms which may be the same or different. Examples of “haloalkyl” include F₃C, ClCH₂, CF₃CH₂ and CF₃CCl₂. The terms “haloalkenyl”, “haloalkynyl”, “haloalkoxy”, “haloalkylthio”, and the like, are defined analogously to the term “haloalkyl”. Examples of “haloalkenyl” include (Cl)₂C═CHCH₂ and CF₃CH₂CH═CHCH₂. Examples of “haloalkynyl” include HC≡CCHCl, CF₃C≡C, CCl₃C≡C and FCH₂C≡CCH₂. Examples of “haloalkoxy” include CF₃O, CCl₃CH₂O, HCF₂CH₂CH₂O and CF₃CH₂O. Examples of “haloalkylthio” include CCl₃S, CF₃S, CCl₃CH₂S and ClCH₂CH₂CH₂S. Examples of “haloalkylsulfinyl” include CF₃S(O), CCl₃S(O), CF₃CH₂S(O) and CF₃CF₂S(O). Examples of “haloalkylsulfonyl” include CF₃S(O)₂, CCl₃S(O)₂, CF₃CH₂S(O)₂ and CF₃CF₂S(O)₂. Examples of “haloalkoxyalkoxy” include CF₃OCH₂O, ClCH₂CH₂OCH₂CH₂O, Cl₃CCH₂OCH₂O as well as branched alkyl derivatives. Examples of “alkylcarbonyl” include C(O)CH₃, C(O)CH₂CH₂CH₃ and C(O)CH(CH₃)₂. Examples of “alkoxycarbonyl” include CH₃OC(═O), CH₃CH₂OC(═O), CH₃ CH₂CH₂OC(═O), (CH₃)₂CHOC(═O) and the different butoxy- or pentoxycarbonyl isomers.

“Aromatic” indicates that each of the ring atoms is essentially in the same plane and has a p-orbital perpendicular to the ring plane, and in which (4n+2) π electrons, when n is 0 or a positive integer, are associated with the ring to comply with Hückel's rule. The term “aromatic carbocyclic ring” includes fully aromatic carbocycles (e.g. phenyl). The term “nonaromatic carbocyclic ring” denotes fully saturated carbocycles as well as partially or fully unsaturated carbocycles where the Hückel rule is not satisfied. The term “hetero” in connection with rings refers to a ring in which at least one ring atom is not carbon and which can contain 1 to 4 heteroatoms independently selected from the group consisting of nitrogen, oxygen and sulfur, provided that each ring contains no more than 4 nitrogens, no more than 2 oxygens and no more than 2 sulfurs. The terms “heteroaromatic ring includes fully aromatic heterocycles. The term “nonaromatic heterocyclic ring” denotes fully saturated heterocycles as well as partially or fully unsaturated heterocycles where the Hückel rule is not satisfied. The heterocyclic ring can be attached through any available carbon or nitrogen by replacement of a hydrogen on said carbon or nitrogen.

One skilled in the art will appreciate that not all nitrogen containing heterocycles can form N-oxides since the nitrogen requires an available lone pair for oxidation to the oxide; one skilled in the art will recognize those nitrogen containing heterocycles which can form N-oxides. One skilled in the art will also recognize that tertiary amines can form N-oxides. Synthetic methods for the preparation of N-oxides of heterocycles and tertiary amines are very well known by one skilled in the art including the oxidation of heterocycles and tertiary amines with peroxy acids such as peracetic and m-chloroperbenzoic acid (MCPBA), hydrogen peroxide, alkyl hydroperoxides such as t-butyl hydroperoxide, sodium perborate, and dioxiranes such as dimethydioxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritkky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzly and A. J. Boulton, Eds., Academic Press.

The total number of carbon atoms in a substituent group is indicated by the “Cᵢ-Cⱼ” prefix where i and j are numbers from 1 to 8. For example, C₁-C₃ alkylsulfonyl designates methylsulfonyl through propylsulfonyl; C₂ alkoxyalkyl designates CH₃OCH₂; C₃ alkoxyalkyl designates, for example, CH₃CH(OCH₃), CH₃OCH₂CH₂ or CH₃CH₂OCH₂; and C₄ alkoxyalkyl designates the various isomers of an alkyl group substituted with an alkoxy group containing a total of four carbon atoms, examples including CH₃CH₂CH₂OCH₂ and CH₃CH₂OCH₂CH₂.

When a compound is substituted with a substituent bearing a subscript that indicates the number of said substituents can exceed 1, said substituents (when they exceed 1) are independently selected from the group of defined substituents. Further, when the subscript indicates a range, e.g. (R)ᵢ₋ⱼ, then the number of substituents may be selected from the integers between i and j inclusive.

The term “optionally substituted with from one to three substituents” and the like indicates that one to three of the available positions on the group may be substituted. When a group contains a substituent which can be hydrogen, for example R¹ or R², then when this substituent is taken as hydrogen, it is recognized that this is equivalent to said group being unsubstituted.

Compounds of Formula I can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. Accordingly, the present invention comprises compounds selected from Formula I, N-oxides and agriculturally suitable salts thereof. The compounds of Formula I may be present as a mixture of stereoisomers, individual stereoisomers, or as an optically active form. In particular, when R¹ and R² of Formula I are different, then said Formula possesses a chiral center at the carbon to which R¹ and R² are commonly bonded.

This invention includes racemic mixtures of equal parts of Formula I′ and Formula I″. 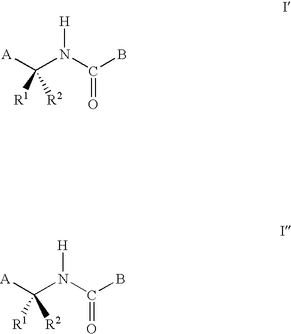
wherein A is a 2-pyridinyl group substituted with (R⁵)ₘ and B is a 3-pyridinyl group substituted with X and either Y or Z, and (R⁶)ₚ, and X, Y or Z, R⁵, R⁶, m and p are as defined above.

In addition, this invention includes compounds and compositions that are enriched compared to the racemic mixture in an enantiomer of the Formula I′ or Formula I″. Included are compounds and compositions involving the essentially pure enantiomers of Formula I′ or Formula I″. For example, this invention includes compounds of Formula I that are enriched in an enantiomer of the Formula I′ compared to the racemic mixture. Included are the essentially pure enantiomers of Formula I′. This invention also includes compositions wherein component (a) is enriched in a component (a) enantiomer of Formula I′ compared to the racemic mixture. This invention also includes compounds of Formula I that are enriched in an enantiomer of the Formula I″ compared to the racemic mixture. Included are the essentially pure enantiomers of Formula I″. This invention also includes compositions wherein component (a) is enriched in a component (a) enantiomer of Formula I″ compared to the racemic mixture

When enantiomerically enriched, one enantiomer is present in greater amounts that the other and the extent of enrichment can be defined by an expression of enantiomer excess(“ee”), which is defined as 100(2x-1) wherein x is the mole fraction of the dominant enantiomer in the enantiomer mixture (e.g., an ee of 20% corresponds to a 60:40 ratio of enantiomers).

The more active enantiomer with respect to the relative positions of R¹, R², A and the rest of the molecule bonded through nitrogen corresponds to the configuration of the enantiomer that, when in a solution of CDCl₃, rotates plane polarized light in the (+) or dextro direction.

Preferably there is at least a 50% enantiomeric excess; more preferably at least a 75% enantiomeric excess; still more preferably at least a 90% enantiomeric excess; and the most preferably at least a 94% enantiomeric excess of the more active isomer of Formula I. Of particular note are enantiomerically pure embodiments of the more active isomer of Formula I.

The salts of the compounds of the invention include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. The salts of the compounds of the invention also include those formed with organic bases (e.g., pyridine, ammonia, or triethylamine) or inorganic bases (e.g., hydrides, hydroxides, or carbonates of sodium, potassium, lithium, calcium, magnesium or barium) when the compound contains an acidic group such as a carboxylic acid or phenol.

As noted above, X and either Y or Z are a linking chain 3 or 4 atoms in length attached to contiguous carbon atoms and are taken together with said carbon atoms to form a fused phenyl ring, a fused 5- or 6-membered nonaromatic carbocyclic or heterocyclic ring optionally including one or two ring members selected from the group consisting of C(═O), SO and S(O)₂, or a fused 5- or 6-membered heteroaromatic ring, each fused ring optionally substituted with one to three substituents independently selected from R⁷. The term “optionally substituted” in connection with these fused rings refers to rings which are unsubstituted or have at least one non-hydrogen substituent that does not extinguish the biological activity possessed by the unsubstituted analog. An example of a fused phenyl ring optionally substituted with one to three substituents independently selected from R⁷ is illustrated as K-38 in Exhibit 1. Examples of 5- or 6-membered heteroaromatic rings optionally substituted with 1 to 3 R⁷ include the rings K-1 through K-37 illustrated in Exhibit 1. Examples of 5- or 6-membered nonaromatic carbocyclic or heterocyclic rings optionally including one or two ring members selected from the group consisting of C(═O), SO and S(O)₂ optionally substituted with 1 to 3 R⁷ include the rings K-39 through K-53 illustrated in Exhibit 1. In these examples, the wavy lines indicate the attachment points of these fused rings to the rest of the molecule of Formula I and n is 0, 1, 2 or 3. The attachment point illustrated at the upper right is the attachment point X and the attachment point illustrated at the lower right is the attachment point Y or the attachment point Z. R¹³ is a subset of R⁷ and is selected from H, C₁-C₄ alkyl or C₁-C₄ haloalkyl. When Y or Z is not used as a ring fusion attachment point, that position is either unsubstituted (i.e. Y or Z is H) or is a group selected from R⁶.

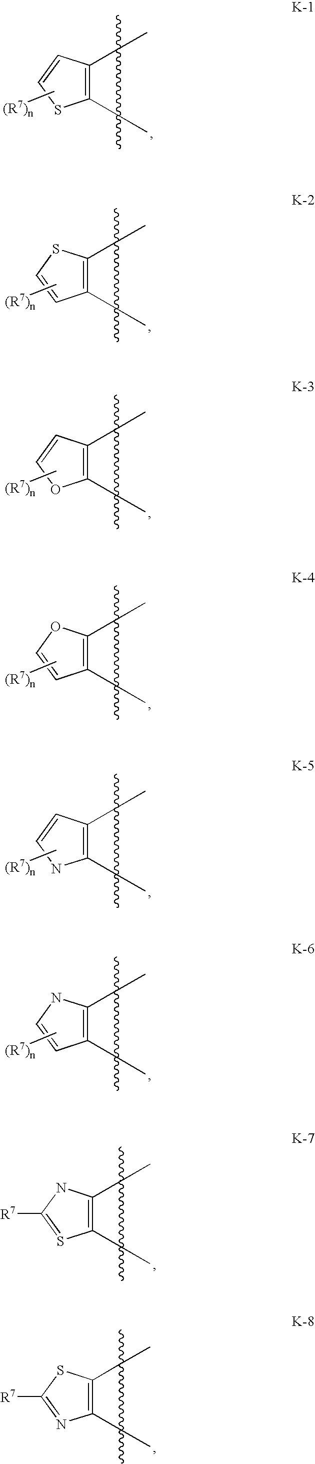

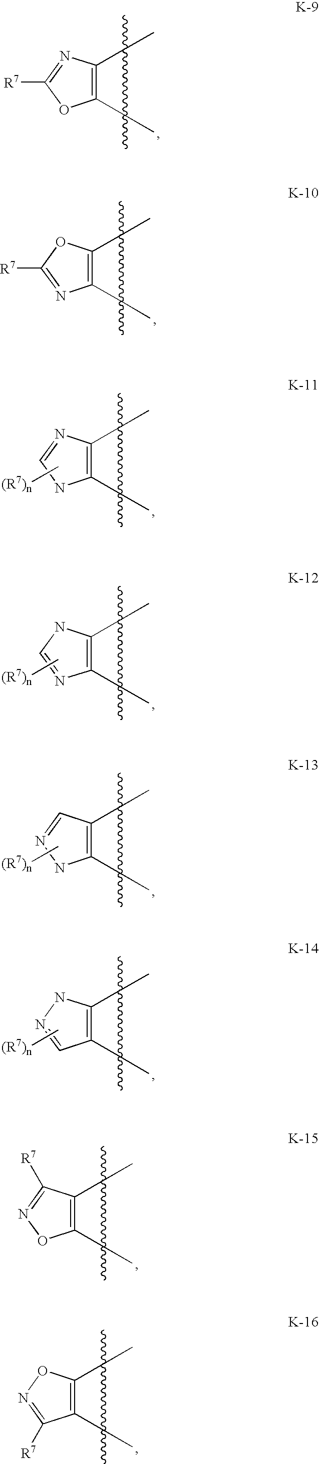

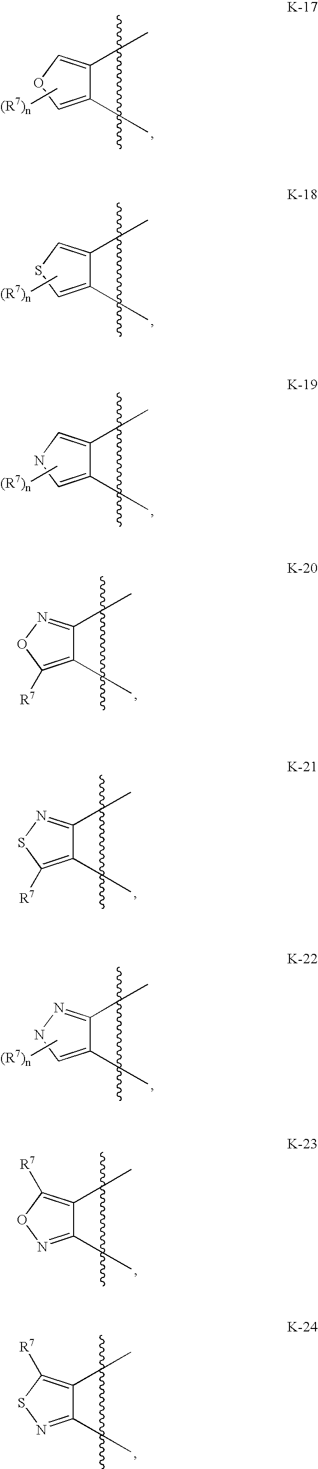

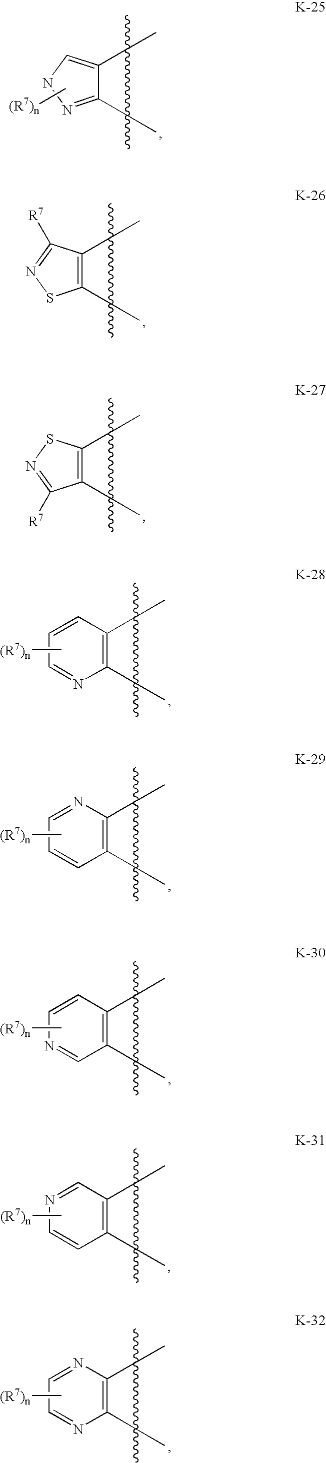

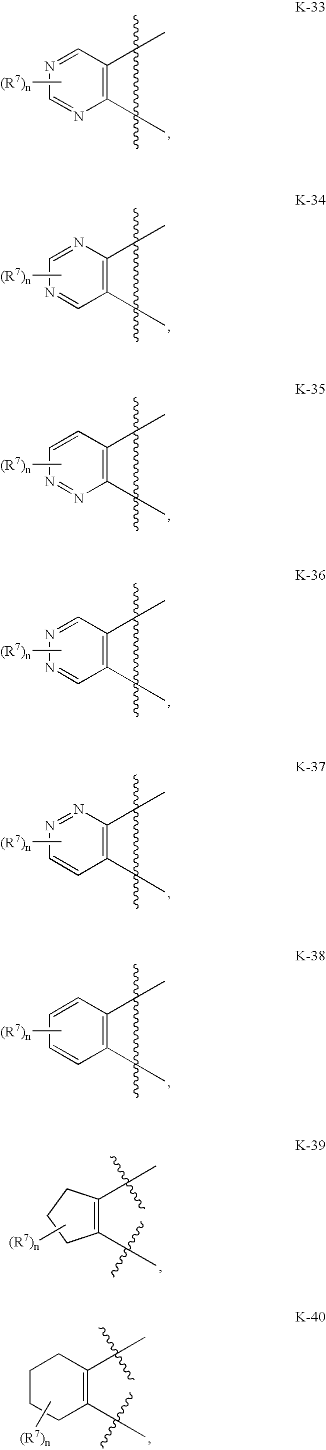

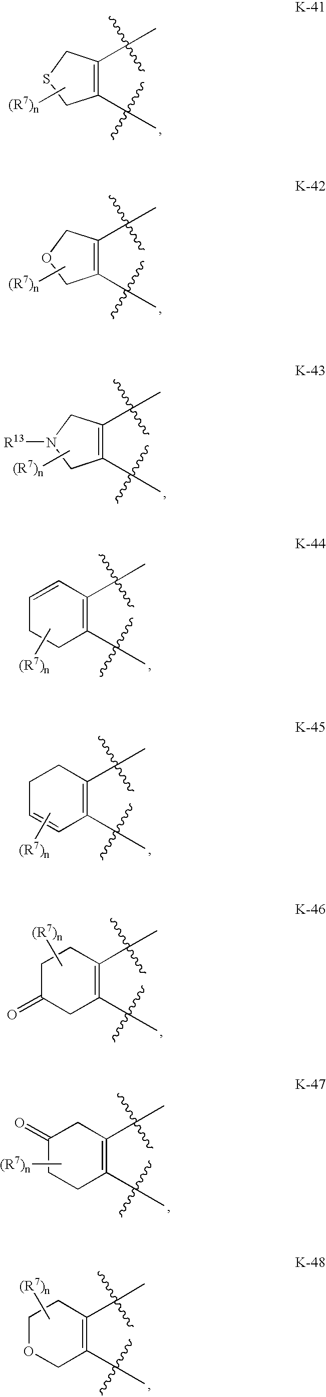

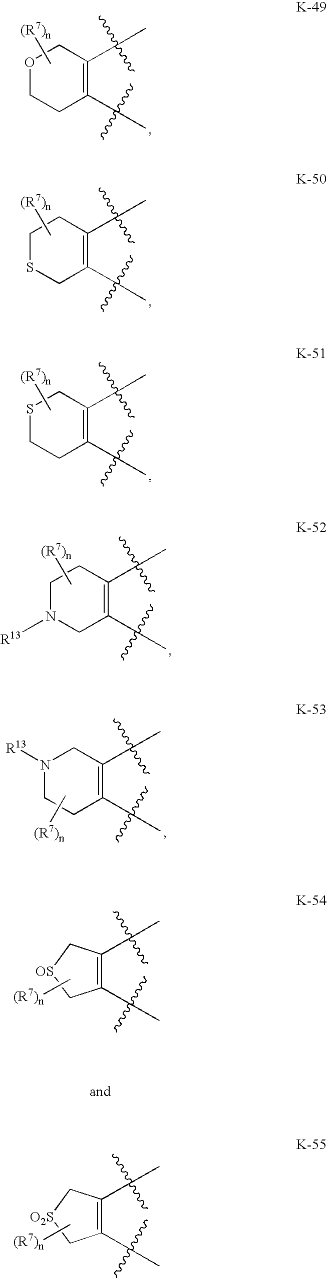

Preferred fused rings are K-38, K-40 and K-2, each fused at the X and Z attachment points.

Preferred compounds for reasons of better activity and/or ease of synthesis are:

Preferred 1. Compounds of Formula I above, an N-oxide or agriculturally suitable salts thereof, wherein X and either Y or Z and the carbon atoms to which they are attached form a fused 5- or 6-membered nonaromatic carbocyclic ring or a fused 5- or 6-membered nonaromatic heterocyclic ring, each fused ring optionally substituted with one to three substituents independently selected from R⁷.

Of note are compounds of Preferred 1 wherein each R⁵ is independently C₁-C₆ alkyl, C₂-C₆ alkenyl, C₂-C₆ alkynyl, C₃-C₆ cycloalkyl, C₁-C₆ haloalkyl, C₂-C₆ haloalkenyl, C₁-C₆ haloalkynyl, C₃-C₆ halocycloalkyl, halogen, CN, CO₂H, CONH₂, NO₂, hydroxy, C₁-C₄ alkoxy, C₁-C₄ haloalkoxy, C₁-C₄ alkylthio, C₁-C₄ alkylsulfinyl, C₁-C₄ alkylsulfonyl, C₁-C₄ haloalkylthio, C₁-C₄ haloalkylsulfinyl, C₁-C₄ haloalkylsulfonyl, C₁-C₄ alkylamino, C₂-C₈ dialkylamino, C₃-C₆ cycloalkylamino, C₂-C₆ alkylcarbonyl, C₂-C₆ alkoxycarbonyl, C₂-C₆ alkylaminocarbonyl, C₃-C₈ dialkylaminocarbonyl or C₃-C₆ trialkylsilyl.

Preferred 2. Compounds of Preferred 1 wherein one R⁵ is in the 3-position and a second R⁵ is in the 5-position and said two R⁵ groups are independently selected from the group consisting of C₁-C₆ alkyl, C₁-C₆ haloalkyl, halogen, CN, C₁-C₄ alkoxy, C₁-C₄ haloalkoxy, C₁-C₄ alkylthio, C₁-C₄ alkylsulfinyl, C₁-C₄ alkylsulfonyl, C₁-C₄ haloalkylthio, C₁-C₄ haloalkylsulfinyl and C₁-C₄ haloalkylsulfonyl.

Of note are compounds of Preferred 2 wherein each R⁵ is independently selected from the group consisting of Cl, Br, I, CH₃, OCF₃, OCHF₂, OCH₂CF₃, OCF₂CF₃, OCF₂CF₂H, OCHFCF₃, SCF₃, SCHF₂, SCH₂CF₃, SCF₂CF₃, SCF₂CF₂H, SCHFCF₃, SOCF₃, SOCHF₂, SOCH₂CF₃, SOCF₂CF₃, SOCF₂CF₂H, SOCHFCF₃, SO₂CF₃, SO₂CHF₂, SO₂CH₂CF₃, SO₂CF₂CF₃, SO₂CF₂CF₂H and SO₂CHFCF₃.

Preferred 3. Compounds of Preferred 2 wherein the R⁵ in the 3-position is selected from halogen and the R⁵ in the 5-position is selected from the group consisting of halogen, C₁-C₆ haloalkyl, C₁-C₄ haloalkoxy, C₁-C₄ haloalkylthio, C₁-C₄ haloalkylsulfinyl and C₁-C₄ haloalkylsulfonyl.

Preferred 4. Preferred are compounds of Preferred 3 wherein the R⁵ in the 3-position is selected from halogen and the R⁵ in the 5-position is selected from the group consisting of halogen, C₁-C₆ haloalcoxy and C₁-C₆ haloalkyl.

Of note are compounds of Preferred 4 wherein the R⁵ in the 3-position is chloro and the R⁵ in the 5-position is trifluoromethyl.

Also of note are compounds of Preferred 4 wherein the R⁵ in the 3-position is chloro and the R⁵ in the 5-position is selected from halogen or C₁-C₆ haloalkoxy.

Preferred compositions of this invention include those of Preferred 1 through Preferred 4 wherein R¹ is H and R² is H or CH₃. More preferred are compositions of Preferred 1 through Preferred 4 wherein R¹ is H and R² is CH₃.

Also preferred are compounds wherein each R⁶ is independently selected from halogen, C₁-C₆ alkyl, C₁-C₆ haloalkyl and C₁-C₄ haloalkoxy,

Specifically preferred is the compound 2,4-dichloro-N-[[3-chloro-5-(trifluoromethyl)-2-pyridinyl]methyl]-5,6,7,8-tetrahydro-3-quinolinecarboxamide.

Also specifically preferred are the compounds
N-[1-5-bromo-3-chloro-2-pyridinyl)ethyl]-3-chloro-4-isoquinolinecarboxamide;
3-bromo-N-[1-(5-bromo-3-chloro-2-pyridiniyl)ethyl]-4-isoquinolinecarboxamide; and
N-[1-(5-bromo-3-chloro-2-pyridinyl)ethyl]-3-fluoro-4-isoquinolinecaiboxamide.

The compounds of Formula I can be prepared by one or more of the following methods and variations as described in Schemes 1-5. The definitions of A, B and R¹ through R⁶ in the compounds of Formulas 1-4 below are as defined above. Compounds of Formula 1a, 1b and 1c are subsets of Formula 1. Compounds of Formulae Ia, Ib and Ic are subsets of the compounds of Formula I, and all substituents for Formulae Ia, Ib and Ic are as defined above for Formula I.

As shown in Scheme 1, the compounds of Formula Ia can be prepared by treating amine salts of Formula 1 with an appropriate acid chloride in an inert solvent with two molar equivalents of a base (e.g. triethylamine or potassium carbonate) present. Suitable solvents are selected from the group consisting of ethers such as tetrahydrofuran, dimethoxyethane, or diethyl ether; hydrocarbons such as toluene or benzene; and halocarbons such as dichloromethane or chloroform.

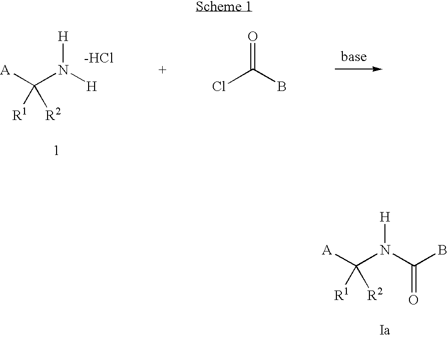

As depicted in Scheme 2, compounds of Formula Ia can be alternatively synthesized by reacting the amine salts of Formula 1 with an appropriate carboxylic acid in the presence of an organic dehydrating reagent such as 1,3-dicyclohexylcarbodiimide (DCC) or 1-[3-(Dirnethylamino)propyl]-3-ethylcarbodiimide hydrochloride (EDC). Suitable solvents are selected from the group consisting of ethers such as tetrahydrofuran, dimethoxyethane, or diethyl ether; hydrocarbons such as toluene or benzene; and halocarbons such as dichloromethane or chloroform. Some acids of Formula B—COOH are know compounds or can be prepared by literature procedures (Tetrahedron Letters 1973, 26, 2335 and Heterocycles 1989, 29(4),707-18)

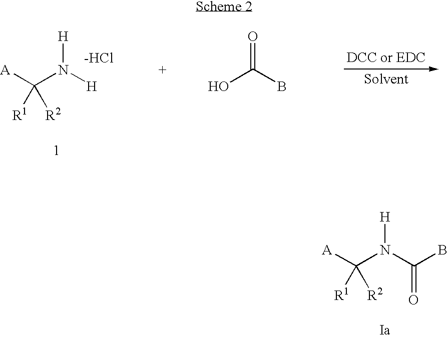

As shown in Scheme 3, the amine salts of Formula 1a, wherein A is 2-pyridyl bearing the indicated substituents and R₁ and R² are hydrogen, can be prepared by reacting the commercially available imine ester 5 with a 2,3-dichloro-pyridine of Formula 4 in the presence of a strong base such as sodium hydride in a polar, aprotic solvent such as N,N-dimethylformamide followed by heating in acidic medium in a procedure analogous to those found in WO99/42447. Compounds of Formula 1b can be prepared by similar procedures in which the intermediate anion resulting from step 1 is treated with an alkylating agent R²—X such as methyl iodide prior to heating in an acidic medium. In the alkylating reagent R²—X, X is a suitable leaving group such as halogen (e.g., Br, I), OS(O)₂CH₃ (methanesulfonate), OS(O)₂CF₃, OS(O)₂Ph-p-CH₃ p-toluenesulfonate), and the like; methanesulfonate works well. Of note are compounds of 1a, 1b and 4 wherein R⁵ is CF₃. Also of note are compounds of 1a, 1b and 4 wherein R⁵ is selected from halogen or C₁-C₆ haloalkoxy.

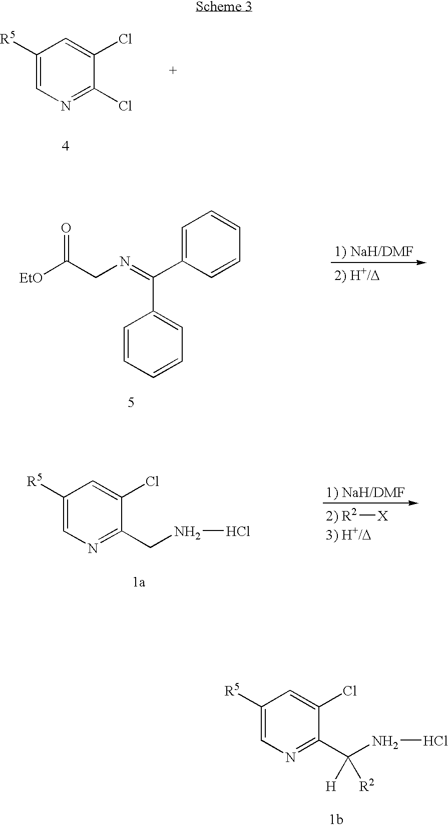

As shown in Scheme 4, compounds of Formula 1c (wherein A is a substituted 2-pyridinyl ring), bearing an aminomethyl group, can be synthesized from nitriles of Formula 2 (wherein A is a substituted 2-pyridinyl ring) by reduction of the nitrile using lithium aluminum hydride (LAH) in toluene.

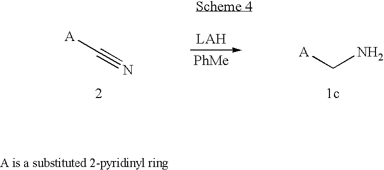

As shown in Scheme 5, compounds of Formula 1c (wherein A is a substituted 2-pyridinyl ring) can be alternatively synthesized by reacting compounds of Formula 3 with ammonia in a protic solvent such as methanol to provide compounds of Formula 1c. Compounds of Formula 1 c can also be prepared by reacting compounds of Formula 3 with a potassium salt of phthalimide followed by reaction with either aminoethanol or hydrazine in an alcohol solvent to provide the desired aminomethyl intermediates of Formula 1c.

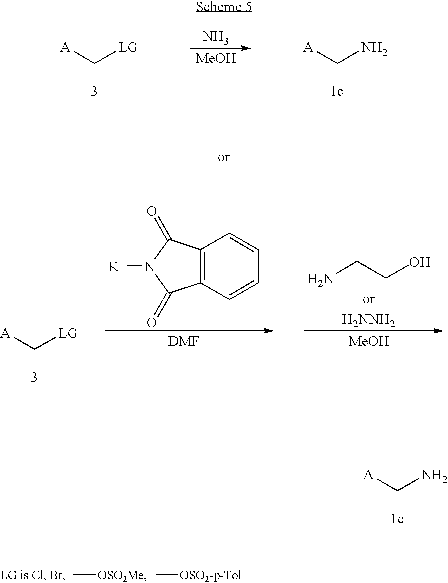

As shown in Scheme 6, carboxylic acids of Formula 8 can be prepared from an aminocarboxylate of Formula 6. Treatment of a compound of Formula 6 with dialkyl malonate followed by halogenation and hydrolysis provides an acid of Formula 8. Further experimental details for this method are described in Example 1. 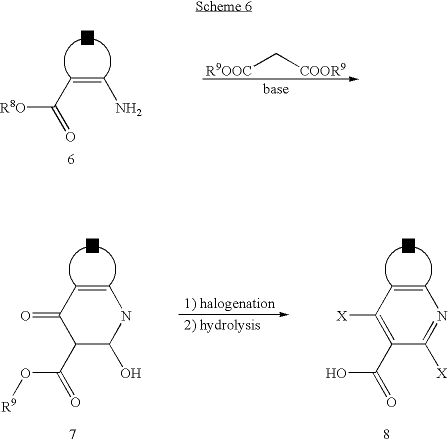
R⁸ and R⁹ are independently C1-C₄ alkyl; and
X is halogen.

It is recognized that some reagents and reaction conditions described above for preparing compounds of Formula I may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after the introduction of a given reagent as it is depicted in any individual scheme, it may be necessary to perform additional routine synthetic steps not described in detail to complete the synthesis of compounds of Formula I. One skilled in the art will also recognize that it may be necessary to perform a combination of the steps illustrated in the above schemes in an order other than that implied by the particular sequence presented to prepare the compounds of Formula I.

One skilled in the art will also recognize that compounds of Formula I and the intermediates described herein can be subjected to various electrophilic, nucleophilic, radical, organometallic, oxidation, and reduction reactions to add substituents or modify existing substituents.

Without further elaboration, it is believed that one skilled in the art using the preceding description can prepare compounds comprising component (a) of the present invention to its fullest extent. The following Example is, therefore, to be construed as merely illustrative, and not limiting of the disclosure in any way whatsoever. Percentages are by weight except for chromatographic solvent mixtures or where otherwise indicated. Parts and percentages for chromatographic solvent mixtures are by volume unless otherwise indicated. ¹H NMR spectra are reported in ppm downfield from tetramethylsilane; s is singlet, d is doublet, t is triplet, q is quartet, m is multiplet, dd is doublet of doublets, dt is doublet of triplets, br s is broad singlet.

### EXAMPLE 1

### Preparation of 2,4-Dichloro-N-[[3-chloro-5-(trifluoromethyl)-2-pyridinyl]methyl]-5,6,7,8-tetrahydro-3-quinolinecarboxamide

### Step A: Preparation of Ethyl 1,4,5,6,7,8-hexahydro-2-hydroxy-4-oxo-3-quinolinecarboxylate

A solution of ethyl 2-amino-1-cyclohexene-1-carboxylate (5.0 g), diethyl malonate (4.7 mL) and sodium ethoxide (2.68 M in ethanol, 12 mL) in ethanol (6 mL) was heated to reflux overnight. The reaction mixture was cooled to room temperature, poured into water and acidified with concentrated HCl to precipitate a tan solid. The solid was filtered, washed with ethyl acetate and dried to yield 630 mg of the title compound.

### Step B: Preparation of Ethyl 2,4-dichloro-5,6,7,8-tetrahydro-3-quinolinecarboxylate

A solution of ethyl 1,4,5,6,7,8-hexahydro-2-hydroxy-4-oxo-3-quinolinecarboxylate (i.e. the product of Step A) (630 mg) in phosphorus oxychloride (10 mL) was refluxed overnight. The reaction mixture was cooled to room temperature and concentrated under reduced pressure. The residue was partitioned between ethyl acetate and water. The organic extracts were dried over magnesium sulfate and concentrated under reduced pressure. The crude product was purified by chromatography on silica gel to give the title compound (580 mg).

### Step C: Preparation of 2,4-Dichloro-5,6,7,8-tetrahydro-3-quinolinecarboxaldehyde

To a solution of ethyl 2,4-dichloro-5,6,7,8-tetrahydro-3-quinolinecarboxylate (i.e. the product from Step B) (450 mg) in 13 mL of dichloromethane at 0° C. was added diisobutylaluminum hydride (1.0 M in dichloromethane, 5 mL). After stirring at 0° C. for 5 hours following by warming up to room temperature overnight, the reaction mixture was added methanol (10 mL) and stirred for additional 30 minutes. The resulting mixture was extracted with ethyl acetate. The extracts were dried over magnesium sulfate and concentrated under reduced pressure to give 346 mg of the title compound as an oil.

### Step D: Preparation of 2,4-Dichloro-5,6,7,8-tetrahydro-3-quinolinecarboxylic acid

A mixture of 2,4-dichloro-5,6,7,8-tetrahydro-3-quinolinecarboxaldehyde (i.e. the product from Step C) (130 mg), sodium chlorite (78 mg) and sulfamic acid (71 mg) in tetrahydrofuran (2 mL) and water (5 mL) was stirred at room temperature for 3 hours. The mixture was then adjusted to a pH=11 by adding IN aqueous NaOH followed by extraction with ethyl acetate. The aqueous layer was then acidified with concentrated HCl to bring the solution to a pH=2 and extracted with ethyl acetate. The organic extracts were dried over magnesium sulfate and concentrated under reduced pressure to give 116 mg of the title compound.

### Step E Preparation of 2,4-Dichloro-N-[[3-chloro-5-(trifluoromethyl -2-pyridinyl]methyl]-5,6,7,8-tetrahydro-3-quinolinecarboxamide

A mixture of 2,4-dichloro-5,6,7,8-tetrahydro-3-quinolinecarboxylic acid (116 mg) (i.e. the product from Step D), oxalyl chloride (358 mg) and 1 drop of N,N-dimethylformamide in 5 mL of dichloromethane was stirred at room temperature for 3 hours. The reaction mixture was then concentrated under reduced pressure to give 124 mg of the corresponding acid chloride intermediate. The crude acid chloride was dissolved in 1 mL of dichloromethane and added to a solution of triethylamine (0.073 mL) and 2-aminomethyl-3-chloro-5-trifluoromethylpyridine hydrochloride (109 mg) (prepared as described in WO99/42447) in dichloromethane (19 mL) at room temperature. After stirring at room temperature overnight, the reaction mixture was washed with 1N aqueous HCl. The organic phase was dried over magnesium sulfate and concentrated under reduced pressure to give an oil. The oil was chromatographed on silica gel using 1:1 hexanes:ethyl acetate as eluent to give 141 mg of the title compound as a yellow solid melting at 48-50° C.

By the procedures described herein together with methods known in the art, the following compounds of Tables 1-2 can be prepared. The following abbreviations are used in the Tables which follow: t is tertiary, s is secondary, n is normal, i is iso, c is cyclo, Me is methyl Et is ethyl, Pr is propyl, i-Pr is isopropyl, Bu is butyl, Ph is phenyl, OMe is methoxy, OEt is ethoxy, SMe is methylthio, SEt is ethylthio, CN is cyano, NO₂ is nitro, TMS is trirethylsilyl, S(O)Me is methylsulfinyl and S(O)₂Me is methylsulfonyl.

TABLE 1

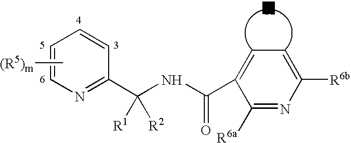

| | | | | | |
|---|---|---|---|---|---|
| R¹ | R² | (R⁵)ₘ | R^{6a} | R^{6b} | R⁷ |

K-1

| | | | | | |
|---|---|---|---|---|---|
| H | H | 3-Cl-5-Br | Cl | Cl | H |
| H | H | 3-Cl-5-Cl | Cl | Cl | H |
| H | H | 3-Cl-5-I | Cl | Cl | H |
| H | H | 3-Cl-5-OCHF₂ | Cl | Cl | H |
| H | H | 3-Cl-5-OCH₂CF₃ | Cl | Cl | H |
| H | H | 3-Cl-5-CF₃ | Cl | Cl | H |
| H | H | 3-Br-5-Br | Cl | Cl | H |
| H | H | 3-Br-5-Cl | Cl | Cl | H |
| H | H | 3-Br-5-I | Cl | Cl | H |
| H | H | 3-Br-5-OCHF₂ | Cl | Cl | H |
| H | H | 3-Br-5-OCH₂CF₃ | Cl | Cl | H |
| H | H | 3-Br-5-CF₃ | Cl | Cl | H |
| H | CH₃ | 3-Cl-5-Br | Cl | Cl | H |
| H | CH₃ | 3-Cl-5-Cl | Cl | Cl | H |
| H | CH₃ | 3-Cl-5-I | Cl | Cl | H |
| H | CH₃ | 3-Cl-5-OCHF₂ | Cl | Cl | H |
| H | CH₃ | 3-Cl-5-OCH₂CF₃ | Cl | Cl | H |
| H | CH₃ | 3-Cl-5-CF₃ | Cl | Cl | H |
| H | CH₃ | 3-Br-5-Br | Cl | Cl | H |
| H | CH₃ | 3-Br-5-Cl | Cl | Cl | H |
| H | CH₃ | 3-Br-5-I | Cl | Cl | H |
| H | CH₃ | 3-Br-5-OCHF₂ | Cl | Cl | H |
| H | CH₃ | 3-Br-5-OCH₂CF₃ | Cl | Cl | H |
| H | CH₃ | 3-Br-5-CF₃ | Cl | Cl | H |
| H | H | 3-Cl-5-Br | Cl | H | H |
| H | H | 3-Cl-5-Cl | Cl | H | H |
| H | H | 3-Cl-5-I | Cl | H | H |
| H | H | 3-Cl-5-OCHF₂ | Cl | H | H |
| H | H | 3-Cl-5-OCH₂CF₃ | Cl | H | H |
| H | H | 3-Cl-5-CF₃ | Cl | H | H |
| H | H | 3-Br-5-Br | Cl | H | H |
| H | H | 3-Br-5-Cl | Cl | H | H |
| H | H | 3-Br-5-I | Cl | H | H |
| H | H | 3-Br-5-OCHF₂ | Cl | H | H |
| H | H | 3-Br-5-OCH₂CF₃ | Cl | H | H |
| H | H | 3-Br-5-CF₃ | Cl | H | H |
| H | CH₃ | 3-Cl-5-Br | Cl | H | H |
| H | CH₃ | 3-Cl-5-Cl | Cl | H | H |
| H | CH₃ | 3-Cl-5-I | Cl | H | H |
| H | CH₃ | 3-Cl-5-OCHF₂ | Cl | H | H |
| H | CH₃ | 3-Cl-5-OCH₂CF₃ | Cl | H | H |
| H | CH₃ | 3-Cl-5-CF₃ | Cl | H | H |
| H | CH₃ | 3-Br-5-Br | Cl | H | H |
| H | CH₃ | 3-Br-5-Cl | Cl | H | H |
| H | CH₃ | 3-Br-5-I | Cl | H | H |
| H | CH₃ | 3-Br-5-OCHF₂ | Cl | H | H |
| H | CH₃ | 3-Br-5-OCH₂CF₃ | Cl | H | H |
| H | CH₃ | 3-Br-5-CF₃ | Cl | H | H |

K-2

| | | | | | |
|---|---|---|---|---|---|
| H | H | 3-Cl-5-Br | Cl | Cl | H |
| H | H | 3-Cl-5-Cl | Cl | Cl | H |
| H | H | 3-Cl-5-I | Cl | Cl | H |
| H | H | 3-Cl-5-OCHF₂ | Cl | Cl | H |
| H | H | 3-Cl-5-OCH₂CF₃ | Cl | Cl | H |
| H | H | 3-Cl-5-CF₃ | Cl | Cl | H |
| H | H | 3-Br-5-Br | Cl | Cl | H |
| H | H | 3-Br-5-Cl | Cl | Cl | H |
| H | H | 3-Br-5-I | Cl | Cl | H |
| H | H | 3-Br-5-OCHF₂ | Cl | Cl | H |
| H | H | 3-Br-5-OCH₂CF₃ | Cl | Cl | H |
| H | H | 3-Br-5-CF₃ | Cl | Cl | H |
| H | CH₃ | 3-Cl-5-Br | Cl | Cl | H |
| H | CH₃ | 3-Cl-5-Cl | Cl | Cl | H |
| H | CH₃ | 3-Cl-5-I | Cl | Cl | H |
| H | CH₃ | 3-Cl-5-OCHF₂ | Cl | Cl | H |
| H | CH₃ | 3-Cl-5-OCH₂CF₃ | Cl | Cl | H |
| H | CH₃ | 3-Cl-5-CF₃ | Cl | Cl | H |
| H | CH₃ | 3-Br-5-Br | Cl | Cl | H |
| H | CH₃ | 3-Br-5-Cl | Cl | Cl | H |
| H | CH₃ | 3-Br-5-I | Cl | Cl | H |
| H | CH₃ | 3-Br-5-OCHF₂ | Cl | Cl | H |
| H | CH₃ | 3-Br-5-OCH₂CF₃ | Cl | Cl | H |
| H | CH₃ | 3-Br-5-CF₃ | Cl | Cl | H |
| H | H | 3-Cl-5-Cl | Cl | H | H |
| H | H | 3-Cl-5-I | Cl | H | H |
| H | H | 3-Cl-5-OCHF₂ | Cl | H | H |
| H | H | 3-Cl-5-OCH₂CF₃ | Cl | H | H |
| H | H | 3-Cl-5-CF₃ | Cl | H | H |
| H | H | 3-Br-5-Br | Cl | H | H |
| H | H | 3-Br-5-Cl | Cl | H | H |
| H | H | 3-Br-5-I | Cl | H | H |
| H | H | 3-Br-5-OCHF₂ | Cl | H | H |
| H | H | 3-Br-5-OCH₂CF₃ | Cl | H | H |
| H | H | 3-Br-5-CF₃ | Cl | H | H |
| H | CH₃ | 3-Cl-5-Br | Cl | H | H |
| H | CH₃ | 3-Cl-5-Cl | Cl | H | H |
| H | CH₃ | 3-Cl-5-I | Cl | H | H |
| H | CH₃ | 3-Cl-5-OCHF₂ | Cl | H | H |
| H | CH₃ | 3-Cl-5-OCH₂CF₃ | Cl | H | H |
| H | CH₃ | 3-Cl-5-CF₃ | Cl | H | H |
| H | CH₃ | 3-Br-5-Br | Cl | H | H |
| H | CH₃ | 3-Br-5-Cl | Cl | H | H |
| H | CH₃ | 3-Br-5-I | Cl | H | H |
| H | CH₃ | 3-Br-5-OCHF₂ | Cl | H | H |
| H | CH₃ | 3-Br-5-OCH₂CF₃ | Cl | H | H |
| H | CH₃ | 3-Br-5-CF₃ | Cl | H | H |

K-3

| | | | | | |
|---|---|---|---|---|---|
| H | H | 3-Cl-5-Br | Cl | Cl | H |
| H | H | 3-Cl-5-Cl | Cl | Cl | H |
| H | H | 3-Cl-5-I | Cl | Cl | H |
| H | H | 3-Cl-5-OCHF₂ | Cl | Cl | H |
| H | H | 3-Cl-5-OCH₂CF₃ | Cl | Cl | H |
| H | H | 3-Cl-5-CF₃ | Cl | Cl | H |
| H | H | 3-Br-5-Br | Cl | Cl | H |
| H | H | 3-Br-5-Cl | Cl | Cl | H |
| H | H | 3-Br-5-I | Cl | Cl | H |
| H | H | 3-Br-5-OCHF₂ | Cl | Cl | H |
| H | H | 3-Br-5-OCH₂CF₃ | Cl | Cl | H |
| H | H | 3-Br-5-CF₃ | Cl | Cl | H |
| H | CH₃ | 3-Cl-5-Br | Cl | Cl | H |
| H | CH₃ | 3-Cl-5-Cl | Cl | Cl | H |
| H | CH₃ | 3-Cl-5-I | Cl | Cl | H |
| H | CH₃ | 3-Cl-5-OCHF₂ | Cl | Cl | H |
| H | CH₃ | 3-Cl-5-OCH₂CF₃ | Cl | Cl | H |
| H | CH₃ | 3-Cl-5-CF₃ | Cl | Cl | H |
| H | CH₃ | 3-Br-5-Br | Cl | Cl | H |
| H | CH₃ | 3-Br-5-Cl | Cl | Cl | H |
| H | CH₃ | 3-Br-5-I | Cl | Cl | H |
| H | CH₃ | 3-Br-5-OCHF₂ | Cl | Cl | H |
| H | CH₃ | 3-Br-5-OCH₂CF₃ | Cl | Cl | H |
| H | CH₃ | 3-Br-5-CF₃ | Cl | Cl | H |
| H | H | 3-Cl-5-Br | Cl | H | H |
| H | H | 3-Cl-5-Cl | Cl | H | H |
| H | H | 3-Cl-5-I | Cl | H | H |
| H | H | 3-Cl-5-OCHF₂ | Cl | H | H |
| H | H | 3-Cl-5-OCH₂CF₃ | Cl | H | H |
| H | H | 3-Cl-5-CF₃ | Cl | H | H |
| H | H | 3-Br-5-Br | Cl | H | H |
| H | H | 3-Br-5-Cl | Cl | H | H |
| H | H | 3-Br-5-I | Cl | H | H |
| H | H | 3-Br-5-OCHF₂ | Cl | H | H |
| H | H | 3-Br-5-OCH₂CF₃ | Cl | H | H |
| H | H | 3-Br-5-CF₃ | Cl | H | H |
| H | CH₃ | 3-Cl-5-Br | Cl | H | H |
| H | CH₃ | 3-Cl-5-Cl | Cl | H | H |
| H | CH₃ | 3-Cl-5-I | Cl | H | H |
| H | CH₃ | 3-Cl-5-OCHF₂ | Cl | H | H |
| H | CH₃ | 3-Cl-5-OCH₂CF₃ | Cl | H | H |
| H | CH₃ | 3-Cl-5-CF₃ | Cl | H | H |
| H | CH₃ | 3-Br-5-Br | Cl | H | H |
| H | CH₃ | 3-Br-5-Cl | Cl | H | H |
| H | CH₃ | 3-Br-5-I | Cl | H | H |
| H | CH₃ | 3-Br-5-OCHF₂ | Cl | H | H |
| H | CH₃ | 3-Br-5-OCH₂CF₃ | Cl | H | H |
| H | CH₃ | 3-Br-5-CF₃ | Cl | H | H |

K-4

| | | | | | |
|---|---|---|---|---|---|
| H | H | 3-Cl-5-Br | Cl | Cl | H |
| H | H | 3-Cl-5-Cl | Cl | Cl | H |
| H | H | 3-Cl-5-I | Cl | Cl | H |
| H | H | 3-Cl-5-OCHF₂ | Cl | Cl | H |
| H | H | 3-Cl-5-OCH₂CF₃ | Cl | Cl | H |
| H | H | 3-Cl-5-CF₃ | Cl | Cl | H |
| H | H | 3-Br-5-Br | Cl | Cl | H |
| H | H | 3-Br-5-Cl | Cl | Cl | H |
| H | H | 3-Br-5-I | Cl | Cl | H |
| H | H | 3-Br-5-OCHF₂ | Cl | Cl | H |
| H | H | 3-Br-5-OCH₂CF₃ | Cl | Cl | H |
| H | H | 3-Br-5-CF₃ | Cl | Cl | H |
| H | CH₃ | 3-Cl-5-Br | Cl | Cl | H |
| H | CH₃ | 3-Cl-5-Cl | Cl | Cl | H |
| H | CH₃ | 3-Cl-5-I | Cl | Cl | H |
| H | CH₃ | 3-Cl-5-OCHF₂ | Cl | Cl | H |
| H | CH₃ | 3-Cl-5-OCH₂CF₃ | Cl | Cl | H |
| H | CH₃ | 3-Cl-5-CF₃ | Cl | Cl | H |
| H | CH₃ | 3-Br-5-Br | Cl | Cl | H |
| H | CH₃ | 3-Br-5-Cl | Cl | Cl | H |
| H | CH₃ | 3-Br-5-I | Cl | Cl | H |
| H | CH₃ | 3-Br-5-OCHF₂ | Cl | Cl | H |
| H | CH₃ | 3-Br-5-OCH₂CF₃ | Cl | Cl | H |
| H | CH₃ | 3-Br-5-CF₃ | Cl | Cl | H |
| H | H | 3-Cl-5-Br | Cl | H | H |
| H | H | 3-Cl-5-Cl | Cl | H | H |
| H | H | 3-Cl-5-I | Cl | H | H |
| H | H | 3-Cl-5-OCHF₂ | Cl | H | H |
| H | H | 3-Cl-5-OCH₂CF₃ | Cl | H | H |
| H | H | 3-Cl-5-CF₃ | Cl | H | H |
| H | H | 3-Br-5-Br | Cl | H | H |
| H | H | 3-Br-5-Cl | Cl | H | H |
| H | H | 3-Br-5-I | Cl | H | H |
| H | H | 3-Br-5-OCHF₂ | Cl | H | H |
| H | H | 3-Br-5-OCH₂CF₃ | Cl | H | H |
| H | H | 3-Br-5-CF₃ | Cl | H | H |
| H | CH₃ | 3-Cl-5-Br | Cl | H | H |
| H | CH₃ | 3-Cl-5-Cl | Cl | H | H |
| H | CH₃ | 3-Cl-5-I | Cl | H | H |
| H | CH₃ | 3-Cl-5-OCHF₂ | Cl | H | H |
| H | CH₃ | 3-Cl-5-OCH₂CF₃ | Cl | H | H |
| H | CH₃ | 3-Cl-5-CF₃ | Cl | H | H |
| H | CH₃ | 3-Br-5-Br | Cl | H | H |
| H | CH₃ | 3-Br-5-Cl | Cl | H | H |
| H | CH₃ | 3-Br-5-I | Cl | H | H |
| H | CH₃ | 3-Br-5-OCHF₂ | Cl | H | H |
| H | CH₃ | 3-Br-5-OCH₂CF₃ | Cl | H | H |
| H | CH₃ | 3-Br-5-CF₃ | Cl | H | H |

K-38

| | | | | | |
|---|---|---|---|---|---|
| H | H | 3-Cl-5-Br | Cl | Cl | H |
| H | H | 3-Cl-5-Cl | Cl | Cl | H |
| H | H | 3-Cl-5-I | Cl | Cl | H |
| H | H | 3-Cl-5-OCHF₂ | Cl | Cl | H |
| H | H | 3-Cl-5-OCH₂CF₃ | Cl | Cl | H |
| H | H | 3-Cl-5-CF₃ | Cl | Cl | H |
| H | H | 3-Br-5-Br | Cl | Cl | H |
| H | H | 3-Br-5-Cl | Cl | Cl | H |
| H | H | 3-Br-5-I | Cl | Cl | H |
| H | H | 3-Br-5-OCHF₂ | Cl | Cl | H |
| H | H | 3-Br-5-OCH₂CF₃ | Cl | Cl | H |
| H | H | 3-Br-5-CF₃ | Cl | Cl | H |
| H | CH₃ | 3-Cl-5-Br | Cl | Cl | H |
| H | CH₃ | 3-Cl-5-Cl | Cl | Cl | H |
| H | CH₃ | 3-Cl-5-I | Cl | Cl | H |
| H | CH₃ | 3-Cl-5-OCHF₂ | Cl | Cl | H |
| H | CH₃ | 3-Cl-5-OCH₂CF₃ | Cl | Cl | H |
| H | CH₃ | 3-Cl-5-CF₃ | Cl | Cl | H |
| H | CH₃ | 3-Br-5-Br | Cl | Cl | H |
| H | CH₃ | 3-Br-5-Cl | Cl | Cl | H |
| H | CH₃ | 3-Br-5-I | Cl | Cl | H |
| H | CH₃ | 3-Br-5-OCHF₂ | Cl | Cl | H |
| H | CH₃ | 3-Br-5-OCH₂CF₃ | Cl | Cl | H |
| H | CH₃ | 3-Br-5-CF₃ | Cl | Cl | H |
| H | H | 3-Cl-5-Br | Cl | H | H |
| H | H | 3-Cl-5-Cl | Cl | H | H |
| H | H | 3-Cl-5-I | Cl | H | H |
| H | H | 3-Cl-5-OCHF₂ | Cl | H | H |
| H | H | 3-Cl-5-OCH₂CF₃ | Cl | H | H |
| H | H | 3-Cl-5-CF₃ | Cl | H | H |
| H | H | 3-Br-5-Br | Cl | H | H |
| H | H | 3-Br-5-Cl | Cl | H | H |
| H | H | 3-Br-5-I | Cl | H | H |
| H | H | 3-Br-5-OCHF₂ | Cl | H | H |
| H | H | 3-Br-5-OCH₂CF₃ | Cl | H | H |
| H | H | 3-Br-5-CF₃ | Cl | H | H |
| H | CH₃ | 3-Cl-5-Br | Cl | H | H |
| H | CH₃ | 3-Cl-5-Cl | Cl | H | H |
| H | CH₃ | 3-Cl-5-I | Cl | H | H |
| H | CH₃ | 3-Cl-5-OCHF₂ | Cl | H | H |
| H | CH₃ | 3-Cl-5-OCH₂CF₃ | Cl | H | H |
| H | CH₃ | 3-Cl-5-CF₃ | Cl | H | H |
| H | CH₃ | 3-Br-5-Br | Cl | H | H |
| H | CH₃ | 3-Br-5-Cl | Cl | H | H |
| H | CH₃ | 3-Br-5-I | Cl | H | H |
| H | CH₃ | 3-Br-5-OCHF₂ | Cl | H | H |
| H | CH₃ | 3-Br-5-OCH₂CF₃ | Cl | H | H |
| H | CH₃ | 3-Br-5-CF₃ | Cl | H | H |

K-40

| | | | | | |
|---|---|---|---|---|---|
| H | H | 3-Cl-5-Br | Cl | Cl | H |
| H | H | 3-Cl-5-Cl | Cl | Cl | H |
| H | H | 3-Cl-5-I | Cl | Cl | H |
| H | H | 3-Cl-5-OCHF₂ | Cl | Cl | H |
| H | H | 3-Cl-5-OCH₂CF₃ | Cl | Cl | H |
| H | H | 3-Cl-5-CF₃ | Cl | Cl | H |
| H | H | 3-Br-5-Br | Cl | Cl | H |
| H | H | 3-Br-5-Cl | Cl | Cl | H |
| H | H | 3-Br-5-I | Cl | Cl | H |
| H | H | 3-Br-5-OCHF₂ | Cl | Cl | H |
| H | H | 3-Br-5-OCH₂CF₃ | Cl | Cl | H |
| H | H | 3-Br-5-CF₃ | Cl | Cl | H |
| H | CH₃ | 3-Cl-5-Br | Cl | Cl | H |
| H | CH₃ | 3-Cl-5-Cl | Cl | Cl | H |
| H | CH₃ | 3-Cl-5-I | Cl | Cl | H |
| H | CH₃ | 3-Cl-5-OCHF₂ | Cl | Cl | H |
| H | CH₃ | 3-Cl-5-OCH₂CF₃ | Cl | Cl | H |
| H | CH₃ | 3-Cl-5-CF₃ | Cl | Cl | H |
| H | CH₃ | 3-Br-5-Br | Cl | Cl | H |
| H | CH₃ | 3-Br-5-Cl | Cl | Cl | H |
| H | CH₃ | 3-Br-5-I | Cl | Cl | H |
| H | CH₃ | 3-Br-5-OCHF₂ | Cl | Cl | H |
| H | CH₃ | 3-Br-5-OCH₂CF₃ | Cl | Cl | H |
| H | CH₃ | 3-Br-5-CF₃ | Cl | Cl | H |
| H | H | 3-Cl-5-Br | Cl | H | H |
| H | H | 3-Cl-5-Cl | Cl | H | H |
| H | H | 3-Cl-5-I | Cl | H | H |
| H | H | 3-Cl-5-OCHF₂ | Cl | H | H |
| H | H | 3-Cl-5-OCH₂CF₃ | Cl | H | H |
| H | H | 3-Cl-5-CF₃ | Cl | H | H |
| H | H | 3-Br-5-Br | Cl | H | H |
| H | H | 3-Br-5-Cl | Cl | H | H |
| H | H | 3-Br-5-I | Cl | H | H |
| H | H | 3-Br-5-OCHF₂ | Cl | H | H |
| H | H | 3-Br-5-OCH₂CF₃ | Cl | H | H |
| H | H | 3-Br-5-CF₃ | Cl | H | H |
| H | CH₃ | 3-Cl-5-Br | Cl | H | H |
| H | CH₃ | 3-Cl-5-Cl | Cl | H | H |
| H | CH₃ | 3-Cl-5-I | Cl | H | H |
| H | CH₃ | 3-Cl-5-OCHF₂ | Cl | H | H |
| H | CH₃ | 3-Cl-5-OCH₂CF₃ | Cl | H | H |
| H | CH₃ | 3-Cl-5-CF₃ | Cl | H | H |
| H | CH₃ | 3-Br-5-Br | Cl | H | H |
| H | CH₃ | 3-Br-5-Cl | Cl | H | H |
| H | CH₃ | 3-Br-5-I | Cl | H | H |
| H | CH₃ | 3-Br-5-OCHF₂ | Cl | H | H |
| H | CH₃ | 3-Br-5-OCH₂CF₃ | Cl | H | H |
| H | CH₃ | 3-Br-5-CF₃ | Cl | H | H |

TABLE 2

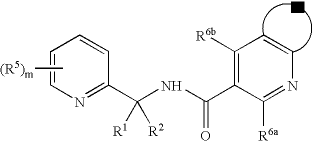

| | | | | | |
|---|---|---|---|---|---|
| R¹ | R² | (R⁵)ₘ | R^{6a} | R^{6b} | R⁷ |

K-1

| | | | | | |
|---|---|---|---|---|---|
| H | H | 3-Cl-5-Br | Cl | Cl | H |
| H | H | 3-Cl-5-Cl | Cl | Cl | H |
| H | H | 3-Cl-5-I | Cl | Cl | H |
| H | H | 3-Cl-5-OCHF₂ | Cl | Cl | H |
| H | H | 3-Cl-5-OCH₂CF₃ | Cl | Cl | H |
| H | H | 3-Cl-5-CF₃ | Cl | Cl | H |
| H | H | 3-Br-5-Br | Cl | Cl | H |
| H | H | 3-Br-5-Cl | Cl | Cl | H |
| H | H | 3-Br-5-I | Cl | Cl | H |
| H | H | 3-Br-5-OCHF₂ | Cl | Cl | H |
| H | H | 3-Br-5-OCH₂CF₃ | Cl | Cl | H |
| H | H | 3-Br-5-CF₃ | Cl | Cl | H |
| H | CH₃ | 3-Cl-5-Br | Cl | Cl | H |
| H | CH₃ | 3-Cl-5-Cl | Cl | Cl | H |
| H | CH₃ | 3-Cl-5-I | Cl | Cl | H |
| H | CH₃ | 3-Cl-5-OCHF₂ | Cl | Cl | H |
| H | CH₃ | 3-Cl-5-OCH₂CF₃ | Cl | Cl | H |
| H | CH₃ | 3-Cl-5-CF₃ | Cl | Cl | H |
| H | CH₃ | 3-Br-5-Br | Cl | Cl | H |
| H | CH₃ | 3-Br-5-Cl | Cl | Cl | H |
| H | CH₃ | 3-Br-5-I | Cl | Cl | H |
| H | CH₃ | 3-Br-5-OCHF₂ | Cl | Cl | H |
| H | CH₃ | 3-Br-5-OCH₂CF₃ | Cl | Cl | H |
| H | CH₃ | 3-Br-5-CF₃ | Cl | Cl | H |

K-2

| | | | | | |
|---|---|---|---|---|---|
| H | H | 3-Cl-5-Br | Cl | Cl | H |
| H | H | 3-Cl-5-Cl | Cl | Cl | H |
| H | H | 3-Cl-5-I | Cl | Cl | H |
| H | H | 3-Cl-5-OCHF₂ | Cl | Cl | H |
| H | H | 3-Cl-5-OCH₂CF₃ | Cl | Cl | H |
| H | H | 3-Cl-5-CF₃ | Cl | Cl | H |
| H | H | 3-Br-5-Br | Cl | Cl | H |
| H | H | 3-Br-5-Cl | Cl | Cl | H |
| H | H | 3-Br-5-I | Cl | Cl | H |
| H | H | 3-Br-5-OCHF₂ | Cl | Cl | H |
| H | H | 3-Br-5-OCH₂CF₃ | Cl | Cl | H |
| H | H | 3-Br-5-CF₃ | Cl | Cl | H |
| H | CH₃ | 3-Cl-5-Br | Cl | Cl | H |
| H | CH₃ | 3-Cl-5-Cl | Cl | Cl | H |
| H | CH₃ | 3-Cl-5-I | Cl | Cl | H |
| H | CH₃ | 3-Cl-5-OCHF₂ | Cl | Cl | H |
| H | CH₃ | 3-Cl-5-OCH₂CF₃ | Cl | Cl | H |
| H | CH₃ | 3-Cl-5-CF₃ | Cl | Cl | H |
| H | CH₃ | 3-Br-5-Br | Cl | Cl | H |
| H | CH₃ | 3-Br-5-Cl | Cl | Cl | H |
| H | CH₃ | 3-Br-5-I | Cl | Cl | H |
| H | CH₃ | 3-Br-5-OCHF₂ | Cl | Cl | H |
| H | CH₃ | 3-Br-5-OCH₂CF₃ | Cl | Cl | H |
| H | CH₃ | 3-Br-5-CF₃ | Cl | Cl | H |

K-3

| | | | | | |
|---|---|---|---|---|---|
| H | H | 3-Cl-5-Br | Cl | Cl | H |
| H | H | 3-Cl-5-Cl | Cl | Cl | H |
| H | H | 3-Cl-5-I | Cl | Cl | H |
| H | H | 3-Cl-5-OCHF₂ | Cl | Cl | H |
| H | H | 3-Cl-5-OCH₂CF₃ | Cl | Cl | H |
| H | H | 3-Cl-5-CF₃ | Cl | Cl | H |
| H | H | 3-Br-5-Br | Cl | Cl | H |
| H | H | 3-Br-5-Cl | Cl | Cl | H |
| H | H | 3-Br-5-I | Cl | Cl | H |
| H | H | 3-Br-5-OCHF₂ | Cl | Cl | H |
| H | H | 3-Br-5-OCH₂CF₃ | Cl | Cl | H |
| H | H | 3-Br-5-CF₃ | Cl | Cl | H |
| H | CH₃ | 3-Cl-5-Br | Cl | Cl | H |
| H | CH₃ | 3-Cl-5-Cl | Cl | Cl | H |
| H | CH₃ | 3-Cl-5-I | Cl | Cl | H |
| H | CH₃ | 3-Cl-5-OCHF₂ | Cl | Cl | H |
| H | CH₃ | 3-Cl-5-OCH₂CF₃ | Cl | Cl | H |
| H | CH₃ | 3-Cl-5-CF₃ | Cl | Cl | H |
| H | CH₃ | 3-Br-5-Br | Cl | Cl | H |
| H | CH₃ | 3-Br-5-Cl | Cl | Cl | H |
| H | CH₃ | 3-Br-5-I | Cl | Cl | H |
| H | CH₃ | 3-Br-5-OCHF₂ | Cl | Cl | H |
| H | CH₃ | 3-Br-5-OCH₂CF₃ | Cl | Cl | H |
| H | CH₃ | 3-Br-5-CF₃ | Cl | Cl | H |

K-4

| | | | | | |
|---|---|---|---|---|---|
| H | H | 3-Cl-5-Br | Cl | Cl | H |
| H | H | 3-Cl-5-Cl | Cl | Cl | H |
| H | H | 3-Cl-5-I | Cl | Cl | H |
| H | H | 3-Cl-5-OCHF₂ | Cl | Cl | H |
| H | H | 3-Cl-5-OCH₂CF₃ | Cl | Cl | H |
| H | H | 3-Cl-5-CF₃ | Cl | Cl | H |
| H | H | 3-Br-5-Br | Cl | Cl | H |
| H | H | 3-Br-5-Cl | Cl | Cl | H |
| H | H | 3-Br-5-I | Cl | Cl | H |
| H | H | 3-Br-5-OCHF₂ | Cl | Cl | H |
| H | H | 3-Br-5-OCH₂CF₃ | Cl | Cl | H |
| H | H | 3-Br-5-CF₃ | Cl | Cl | H |
| H | CH₃ | 3-Cl-5-Br | Cl | Cl | H |
| H | CH₃ | 3-Cl-5-Cl | Cl | Cl | H |
| H | CH₃ | 3-Cl-5-I | Cl | Cl | H |
| H | CH₃ | 3-Cl-5-OCHF₂ | Cl | Cl | H |
| H | CH₃ | 3-Cl-5-OCH₂CF₃ | Cl | Cl | H |
| H | CH₃ | 3-Cl-5-CF₃ | Cl | Cl | H |
| H | CH₃ | 3-Br-5-Br | Cl | Cl | H |
| H | CH₃ | 3-Br-5-Cl | Cl | Cl | H |
| H | CH₃ | 3-Br-5-I | Cl | Cl | H |
| H | CH₃ | 3-Br-5-OCHF₂ | Cl | Cl | H |
| H | CH₃ | 3-Br-5-OCH₂CF₃ | Cl | Cl | H |
| H | CH₃ | 3-Br-5-CF₃ | Cl | Cl | H |

K-38

| | | | | | |
|---|---|---|---|---|---|
| H | H | 3-Cl-5-Br | Cl | Cl | H |
| H | H | 3-Cl-5-Cl | Cl | Cl | H |
| H | H | 3-Cl-5-I | Cl | Cl | H |
| H | H | 3-Cl-5-OCHF₂ | Cl | Cl | H |
| H | H | 3-Cl-5-OCH₂CF₃ | Cl | Cl | H |
| H | H | 3-Cl-5-CF₃ | Cl | Cl | H |
| H | H | 3-Br-5-Br | Cl | Cl | H |
| H | H | 3-Br-5-Cl | Cl | Cl | H |
| H | H | 3-Br-5-I | Cl | Cl | H |
| H | H | 3-Br-5-OCHF₂ | Cl | Cl | H |
| H | H | 3-Br-5-OCH₂CF₃ | Cl | Cl | H |
| H | H | 3-Br-5-CF₃ | Cl | Cl | H |
| H | CH₃ | 3-Cl-5-Br | Cl | Cl | H |
| H | CH₃ | 3-Cl-5-Cl | Cl | Cl | H |
| H | CH₃ | 3-Cl-5-I | Cl | Cl | H |
| H | CH₃ | 3-Cl-5-OCHF₂ | Cl | Cl | H |
| H | CH₃ | 3-Cl-5-OCH₂CF₃ | Cl | Cl | H |
| H | CH₃ | 3-Cl-5-CF₃ | Cl | Cl | H |
| H | CH₃ | 3-Br-5-Br | Cl | Cl | H |
| H | CH₃ | 3-Br-5-Cl | Cl | Cl | H |
| H | CH₃ | 3-Br-5-I | Cl | Cl | H |
| H | CH₃ | 3-Br-5-OCHF₂ | Cl | Cl | H |
| H | CH₃ | 3-Br-5-OCH₂CF₃ | Cl | Cl | H |
| H | CH₃ | 3-Br-5-CF₃ | Cl | Cl | H |

K-40

| | | | | | |
|---|---|---|---|---|---|
| H | H | 3-Cl-5-Br | Cl | Cl | H |
| H | H | 3-Cl-5-Cl | Cl | Cl | H |
| H | H | 3-Cl-5-I | Cl | Cl | H |
| H | H | 3-Cl-5-OCHF₂ | Cl | Cl | H |
| H | H | 3-Cl-5-OCH₂CF₃ | Cl | Cl | H |
| H | H | 3-Cl-5-CF₃ | Cl | Cl | H |
| H | H | 3-Br-5-Br | Cl | Cl | H |
| H | H | 3-Br-5-Cl | Cl | Cl | H |
| H | H | 3-Br-5-I | Cl | Cl | H |
| H | H | 3-Br-5-OCHF₂ | Cl | Cl | H |
| H | H | 3-Br-5-OCH₂CF₃ | Cl | Cl | H |
| H | H | 3-Br-5-CF₃ | Cl | Cl | H |
| H | CH₃ | 3-Cl-5-Br | Cl | Cl | H |
| H | CH₃ | 3-Cl-5-Cl | Cl | Cl | H |
| H | CH₃ | 3-Cl-5-I | Cl | Cl | H |
| H | CH₃ | 3-Cl-5-OCHF₂ | Cl | Cl | H |
| H | CH₃ | 3-Cl-5-OCH₂CF₃ | Cl | Cl | H |
| H | CH₃ | 3-Cl-5-CF₃ | Cl | Cl | H |
| H | CH₃ | 3-Br-5-Br | Cl | Cl | H |
| H | CH₃ | 3-Br-5-Cl | Cl | Cl | H |
| H | CH₃ | 3-Br-5-I | Cl | Cl | H |
| H | CH₃ | 3-Br-5-OCHF₂ | Cl | Cl | H |
| H | CH₃ | 3-Br-5-OCH₂CF₃ | Cl | Cl | H |
| H | CH₃ | 3-Br-5-CF₃ | Cl | Cl | H |

This invention also relates to fungicidal compositions comprising fungicidally effective amounts of a composition of the compounds of the invention and at least one additional component selected from the group consisting of surfactants, solid diluents, liquid diluents and other fungicides. Included are fungicidal compositions comprising a fungicidally effective amount of at least one compound of Formula I and at least one other fungicide.

Of note are compositions comprising (a) at least one compound of Formula I; and
(b) at least one compound selected from the group consisting of
(b1) alkylenebis(dithiocarbamate)fungicides;
(b2) compounds acting at the bc₁ complex of the fungal mitochondrial respiratory electron transfer site;
(b3) cymoxanil;
(b4) compounds acting at the demethylase enzyme of the sterol biosynthesis pathway;
(b5) morpholine and piperidine compounds that act on the sterol biosynthesis pathway;
(b6) phenylamide fungicides;
(b7) pyrimidinone fungicides;
(b8) phthalimides; and
(b9) fosetyl-aluminum.

The weight ratios of component (b) to component (a) typically is from 100:1 to 1:100, preferably is from 30:1 to 1:30, and more preferably is from 10:1 to 1:10. Of note are compositions wherein the weight ratio of component (b) to component (a) is from 10:1 to 1:1. Included are compositions wherein the weight ratio of component (b) to component (a) is from 9:1 to 4.5:1.

The bc₁ Complex Fungicides (component (b2)

Strobilurin fungicides such as azoxystrobin, kresoxim-methyl, metominostrobin/fenominostrobin (SSF-126), picoxystrobin, pyraclostrobin and trifloxystrobin are known to have a fungicidal mode of action which inhibits the bc₁ complex in the mitochondrial respiration chain (Angew. Chem. Int. Ed., 1999, 38, 1328-1349). Methyl (E)-2-[[6-(2-cyanophenoxy)-4pyrimidinyl]oxy]-α-(methoxyimino)benzeneacetate (also known as azoxystrobin) is described as a bc₁ complex inhibitor in Biochemical Society Transactions 1993, 22, 68S. Methyl (E)-α-(methoxyimino)-2-[(2-methylphenoxy)methyl]benzeneacetate (also known as kresoxim-methyl) is described as a bc₁ complex inhibitor in Biochemical Society Transactions 1993, 22, 64S. (E)-2-[(2,5-Dimethylphenoxy)methyl]-α-(methoxyimino)-N-methylbenzeneacetamide is described as a bc₁ complex inhibitor in Biochemistry and Cell Biology 1995, 85(3), 306-311. Other compounds that inhibit the bc₁ complex in the mitochondrial respiration chain include famoxadone and fenamidone.

The bc₁ complex is sometimes referred to by other names in the biochemical literature, including complex III of the electron transfer chain, and ubihydroquinone:cytochrome c oxidoreductase. It is uniquely identified by the Enzyme Commission number EC1.10.2.2. The bc₁ complex is described in, for example, J. Biol. Chem. 1989, 264, 14543-38; Methods Enzymol. 1986, 126, 253-71; and references cited therein.

The Sterol Biosynthesis Inhibitor Fungicides (component (b4) or (b5))

The class of sterol biosynthesis inhibitors includes DMI and non-DMI compounds, that control fungi by inhibiting enzymes in the sterol biosynthesis pathway. DM fungicides have a common site of action within the fungal sterol biosynthesis pathway; that is, an inhibition of demethylation at position 14 of lanosterol or 24-methylene dihydrolanosterol, which are precursors to sterols in fungi. Compounds acting at this site are often referred to as demethylase inhibitors, DMI fungicides, or DMIs. The demethylase enzyme is sometimes referred to by other names in the biochemical literature, including cytochrome P450 (14DM). The demethylase enzyme is described in, for example, J. Biol. Chem. 1992, 267, 13175-79 and references cited therein. DMI fungicides fall into several classes: azoles (including tiazoles and imidazoles), pyrimidines, piperazines and pyridines. The triazoles includes bromuconazole, cyproconazole, difenoconazole, diniconazole, epxiconazole, fenbuconazole, fluquinconazole, flusilazole, flutriafol, hexaconazole, ipconazole, metconazole, penconazole, propiconazole, tebuconazole, tetraconazole, triadimefon, triadlimenol, triticonazole and uniconazole. The imidazoles include clotimazole, econazole, imazalil, isoconazole, miconazole and prochloraz. The pyrimidines include fenarimol, nuarimol and triarimol. The piperazines include triforine. The pyridines include buthiobate and pyrifenox. Biochemical investigations have shown that all of the above mentioned fungicides are DMI fungicides as described by K. H. Kuck, et al. in Modern Selective Fungicides—Properties, Applications and Mechanisms of Action, Lyr, H., Ed.; Gustav Fischer Verlag: New York, 1995, 205-258.

The DMI fungicides have been grouped together to distinguish them from other sterol biosynthesis inhibitors, such as, the morpholine and piperidine fungicides. The morpholines and piperidines are also sterol biosynthesis inhibitors but have been shown to inhibit later steps in the sterol biosynthesis pathway. The morpholines include aldimorph, dodemorph, fenpropimorph, tridemorph and trimorphamide. The piperidines include fenpropidin Biochemical investigations have shown that all of the above mentioned morpholine and piperidine fungicides are sterol biosynthesis inhibitor fungicides as described by K. H. Kuck, et al. in Modern Selective Fungicides—Properties, Applications and Mechanisms of Action, Lyr, H., Ed.; Gustav Fischer Verlag: New York, 1995, 185-204.

Pyrimidinone Fungicides (component (b7))

Pyrimidinone fungicides include compounds of Formula II 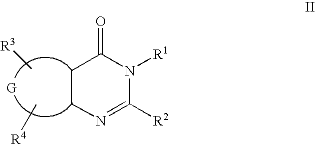
wherein
G is a fused phenyl, thiophene or pyridine ring;
R¹ is C₁-C₆ alkyl;
R² is C₁-C₆ alkyl or C₁-C₆ alkoxy;
R³ is halogen; and
R⁴ is hydrogen or halogen.

Pyrimidinone fungicides are described in International Patent Application WO94/26722, U.S. Pat. No.6,066,638, U.S. Pat. No.6,245,770, U.S. Pat. No. 6,262,058 and U.S. Pat. No.6,277,858.

Of note are pyrimidinone fungicides selected from the group:
6-bromo-3-propyl-2-propyloxy-4(3H)-quinazolinone,
6,8 -diiodo-3-propyl-2-propyloxy-4(3H)-quinazolinone,
6-iodo-3-propyl-2-propyloxy-4(3h)-quinazolinone,
6-chloro-2-propoxy-3-propylthieno[2,3-d]pyrimidin-4(3H)-one,
6-bromo-2-propoxy-3-propylthieno[2,3-d]pyrimidin-4(3H)-one,
7-bromo-2-propoxy-3-propylthieno[3,2-d]pyrimidin-4(3H)-one,
6-bromo-2-propoxy-3-propylpyrido[2,3-d]pyrimidin-4(3H)-one,
6,7-dibromo-2-propoxy-3-propylthieno[3,2-d]pyrimidin-4(3H)-one, and

3-(cyclopropylmethyl)-6-iodo-2-(propylthio)pyrido[2,3-d]pyrimidin-4(3H)-one.

TABLE 8

| | Examples of component (b) |
|---|---|
| (b1) | Alkylenebis(dithiocarbamate)s such as mancozeb, maneb, propineb |
| | and zineb |
| (b3) | Cymoxanil |
| (b6) | Phenylamides such as metalaxyl, benalaxyl and oxadixyl |
| (b8) | Phthalimids such as folpet or captan |
| (b9) | Fosetyl-aluminum |

Preferred 5. Preferred compositions comprise a compound of component (a) mixed with cymoxanil.

Preferred 6. Preferred compositions comprise a compound of component (a) mixed with a compound selected from (b1). More preferred is a composition wherein the compound of (b1) is mancozeb.

Preferred 7. Preferred compositions comprise a compound of component (a) mixed with a compound selected from (b2). More preferred is a composition wherein the compound of (b2) is famoxadone.

Preferred compositions comprise a compound of component (a) mixed with two compounds selected from two different groups selected from (b1), (b2), (b3), (b4), (b5), (b6), (b7), (b8) and (b9).

Preferred compositions are those wherein component (a) is selected from the compounds of Formula I preferred above.

Other fungicides that can be included in compositions of this invention in combination with a Formula I compound or as an additional component in combination with component (a) and component (b) are acibenzolar, benalaxyl, benomyl blasticidin-S, Bordeaux mixture (tribasic copper sulfate), carpropamid, captafol, captan, carbendazim, chloroneb, chlorothalonil, copper oxychloride, copper salts such as copper sulfate and copper hydroxide, cyazofamid, cymoxanil, cyprodinil, (S)-3,5-dichloro-N-(3-chloro-1-ethyl-1-methyl-2-oxopropyl)-4-methylbenzamide (RH 7281), diclocymet (S-2900), diclomezine, dicloran, dimethomorph, diniconazole-M, dodemorph, dodine, edifenphos, fencaramid (SZX0722), fenpiclonil, fentin acetate, fentin hydroxide, fluazinam, fludioxonil, flumetover (RPA 403397), flutolanil, folpet, fosetyl-aluminum, furalaxyl furametapyr (S-82658), iprobenfos, iprodione, isoprothiolane, iprovalicarb, kasugamycin, mancozeb, maneb, mefenoxarn, mepronil, metalaxyl, metiram-zinc, myclobutanil, neo-asozin (ferric methanearsonate), oxadixyl, pencycuron, prochloraz, procymidone, propamocarb, propineb, pyrifenox, pyrimethanil, pyroquilon, quinoxyfen, spiroxamine, sulfur, thifluzamide, thiophanate-methyl, thiram, triadimefon, tricyclazole, validamrycin, vinclozolin, zineb and zoxamid.

Descriptions of the commercially available compounds listed above may be found in The Pesticide Manual, Twelfth Edition, C. D. S. Tomlin, ed., British Crop Protection Council, 2000.

Of note are combinations of Formula I with fungicides of a different biochernical mode of action (e.g. mitochondrial respiration inhibition, inhibition of protein synthesis by interference of the synthesis of ribosomal RNA or inhibition of beta-tubulin synthesis) that can be particularly advantageous for resistance management. Examples include combinations of compounds of Formula I (e.g. Compound 1) with strobilurins such as azoxystrobin, kresoxim-methyl, pyraclostrobin and trifloxystrobin; carbendazim, mitochondrial respiration inhibitors such as famoxadone and fenamidone; benomyl, cymoxanil; dimethomorph; folpet; fosetyl-aluminum; metalaxyl; mancozeb and maneb. These combinations can be particularly advantageous for resistance management, especially where the fungicides of the combination control the same or similar diseases.

Of note are combinations of Formula I with fungicides for controlling grape diseases (e.g. Plasmopara viticola, Botiytis cinerea and Uncinula necatzu) including alkylenebis(dithiocarbamate)s such as mancozeb, maneb, propineb and zineb, phthalimids such as folpet, copper salts such as copper sulfate and copper hydroxide, strobilurins such as azoxystrobin, pyraclostrobin and trifloxystrobin, mitochondrial respiration inhibitors such as famoxadone and fenamidone, phenylamides such as metalaxyl phosphonates such as fosetyl-Al, dimethomorph, pyrimidinone fungicides such as 6-iodo-3-propyl-2-propyloxy-4(3H)-quinazolinone and 6-chloro-2-propoxy-3-propylthieno[2,3-d]pyrimidin-4(3H)-one, and other fungicides such as cymoxanil.

Of note are combinations of Formula I with fungicides for controlling potato diseases (e.g. Phytophthora inifestans, Alternaria solani and Rhizoctonia solani) including alkylenebis(dithiocarbamate)s such as mancozeb, maneb, propineb and zineb; copper salts such as copper sulfate and copper hydroxide; strobilurins such as pyraclostrobin and trifloxystrobin; mitochondrial respiration inhibitors such as famoxadone and fenamidone; phenylamides such as metalaxyl; carbamates such as propamocarb; phenylpyridylamines such as fluazinam and other fungicides such as chlorothalonil, cyazofamid, cymoxanil, dimethomorph, zoxamid and iprovalicarb.

Of note are compositions. wherein component (b) comprises at least one compound from each of two different groups selected from (b1), (b2), (b3), (b4), (b5), (b6), (b7), (b8) and (b9). The weight ratio of the compound(s) of the first of these two component (b) groups to the compound(s) of the second of these component (b) groups typically is from 100:1 to 1:100, more typically from 30:1 to 1:30 andmosttypically from 10:1 to 1:10.

Of note are compositions wherein component (b) comprises at least one compound selected from (b1), for example mancozeb, and at least one compound selected from a second component (b) group, for example, from (b2), (b3), (b6), (b7), (b8) or (b9). Of particular note are such compositions wherein the overall weight ratio of component (b) to component (a) is from 30:1 to 1:30 and the weight ratio of component (b1) to component (a) is from 10:1 to 1:1. Included are compositions wherein the weight ratio of component (b1) to component (a) is fiom 9:1 to 4.5:1. Examples of these compositions include compositions comprising rnixtures of component (a) (preferably a compound from Index Table A and B) with mancozeb and a compound selected from the group consisting of famoxadone, fenamidone, azoxystrobin, kresoxim-methyl, pyraclostrobin, trifloxystrobin, cymoxanil metalaxyl, benalaxyl, oxadixyl 6-iodo-3-propyl-2-propyloxy-4(3H)-quinaiolinone, 6-chloro-2-propoxy-3-propylthieno[2,3-d]pyrimidin-4(3H)-one, folpet, captan and fosetyl-aluminum.

Also of note are compositions wherein component (b) comprises at least one compound selected from (b2), for example famoxadone, and at least one compound selected from a second component (b) group, for example, from (b1), (b3), (b6), (b7), (b8) or (b9). Of particular note are such compositions wherein the overall weight ratio of component (b) to component (a) is from 30:1 to 1:30 and the weight ratio of component (b2) to component (a) is from 10:1 to 1:1. Included are compositions wherein the weight ratio of component (b2) to component (a) is from 9:1 to 4.5:1. Examples of these compositions include compositions comprising mixtures of component (a) (preferably a compound from Index Table A and B) with famoxadone and a compound selected from the group consisting of mancozeb, maneb, propineb, zineb, cymoxanil, metalaxyl benalaxyl, oxadixyl, 6-iodo-3-propyl-2-propyloxy-4(3H)-quinazolinone, 6-chloro-2-propoxy-3-propylthieno[2,3-d]pyrimidin-4(3H)-one, folpet, captan and fosetyl-aluminum.

Also of note are compositions wherein component (b) comprises the compound of (b3), in other words cymoxanil, and at least one compound selected from a second component (b) group, for example, from (b1), (b2), (b6), (b7), (b8) or (b9). Of particular note are such compositions wherein the overall weight ratio of component (b) to component (a) is from 30:1 to 1:30 and the weight ratio of component (b3) to component (a) is from 10:1 to 1:1. Included are compositions wherein the weight ratio of component (b3) to component (a) is from 9:1 to 4.5:1. Examples of these compositions include compositions comprising mixtures of component (a) (preferably a compound from Index Table A and B) with cymoxanil and a compound selected from the group consisting of famoxadone, fenamidone, azoxystrobin, kresoxim-methyl, pyraclostrobin, trifloxystrobin, mancozeb, maneb, propineb, zineb, metalaxyl, benalaxyl, oxadixyl, 6-iodo-3-propyl-2-propyloxy-4(3H)-quinazolinone, 6-chloro-2-propoxy-3-propylthieno[2,3-d]pyrimidin-4(3H)-one, folpet, captan and fosetyl-aluminum.

Also of note are compositions wherein component (b) comprises at least one compound selected from (b6), for example metalaxyl, and at least one compound selected from a second component (b) group, for example, from (b1), (b2), (b3), (b7), (b8) or (b9). Of particular note are such compositions wherein the overall weight ratio of component (b) to component (a) is from 30:1 to 1:30 and the weight ratio of component (b6) to component (a) is from 10:1 to 1:3. Included are compositions wherein the weight ratio of component (b6) to component (a) is from 9:1 to 4.5:1. Examples of these compositions include compositions comprising mixtures of component (a) (preferably a compound from Index Table A and B) with metalaxyl or oxadixyl and a compound selected from the group consisting of famoxadone, fenamidone, azoxystrobin, kresoxim-methyl, pyraclostrobin, trifloxystrobin, cymoxanil, mancozeb, maneb, propineb, zineb, 6-iodo-3-propyl-2-propyloxy-4(3H)-quinazolinone, 6-chloro-2-propoxy-3-propylthieno[2,3-d]pyrimidin-4(3H)-one, folpet, captan and fosetyl-aluminuim.

Also of note are compositions wherein component (b) comprises at least one compound selected from (b7), for example 6-iodo-3-propyl-2-propyloxy-4(3H)-quinazolinone or 6-chloro-2-propoxy-3-propylthieno[2,3-d]pyrimidin-4(3H)-one, and at least one compound selected from a second component (b) group, for example, from (b1), (b2), (b3), (b6), (b8) or (b9). Of particular note are such compositions wherein the overall weight ratio of component (b) to component (a) is from 30:1 to 1:30 and the weight ratio of component (b7) to component (a) is from 1:1 to 1:20. Included are compositions wherein the weight ratio of component (b6) to component (a) is from 1:4.5 to 1:9. Examples of these compositions include compositions comprising mixtures of component (a) (preferably a compound firom Index Table A and B) with 6-iodo-3-propyl-2-propyloxy-4(3H)-quinazolinone or 6-chloro-2-propoxy-3-propylthieno[2,3-d]pyrimidin-4(3H)-one and a compound selected from the group consisting of famoxadone, fenamidone, azoxystrobin, kresoxim-methyl, pyraclostrobin, trifloxystrobin, cymoxanil, mancozeb, maneb, propineb, zineb, metalaxyl, benalaxyl, oxadixyl, folpet, captan and fosetyl-aluminum.

Also of note are compositions wherein component (b) comprises the compound of (b9), in other words fosetyl-aluminum, and at least one compound selected from a second component (b) group, for example, from (b1), (b2), (b3), (b6) or (b7). Of particular note are such compositions wherein the overall weight ratio of component (b) to component (a) is from 30:1 to 1:30 and the weight ratio of component (b9) to component (a) is from 10:1 to 1:1. Included are compositions wherein the weight ratio of component (b9) to component (a) is fiom 9:1 to 4.5:1. Examples ofthese compositions include compositions comprising mixtures of component (a) (preferably a compound from Index Table A and B) with fosetyl-aluminum and a compound selected from the group consisting of famoxadone, fenamidone, azoxystrobin, kresoxim-methyl, pyraclostrobin, trifloxystrobin, mancozeb, maneb, propineb, zineb, metalaxyl, benalaxyl, oxadixyl 6-iodo-3-propyl-2-propyloxy-4(3H)-quinazolinone, 6-chloro-2-propoxy-3-propylthieno[2,3-d]pyrimidin-4(3H)-one, folpet, captan and cymoxanil.

Of note are combinations of compounds of Formula I with fungicides giving an even broader spectrum of agricultural protection including strobilurins such as azoxystrobin, kresoxim-methyl, pyraclostrobin and trifloxystrobin; morpholines such as fenpropidine and fenpropimorph; triazoles such as bromuconazole, cyproconazole, difenoconazole, epoxyconazole, flusilazole, ipconazole, metconazole, propiconazole, tebuconazole and triticonazole; pyrimidinone fungicides, benomyl; carbendazim; chlorothalonil; dimethomorph; folpet; mancozeb; maneb; quinoxyfen; validamycin and vinclozolin.

Of particular note are combinations of Compound 3, Compound 4, Compound 5 or Compound 6 with azoxystrobin, combinations of Compound 3, Compound 4, Compound 5 or Compound 6 with kresoxim-methyl, combinations of Compound 3, Compound 4, Compound 5 or Compound 6 with pyrclostrobin, combinations of Compound 3, Compound 4, Compound 5 or Compound 6 with trifloxystrobin, combinations of Compound 3, Compound 4, Compound 5 or Compound 6 with carbendazim, combinations of Compound 3, Compound 4, Compound 5 or Compound 6 with chlorothalonil, combinations of Compound 3, Compound 4, Compound 5 or Compound 6 with dimethomorph, combinations of Compound 3, Compound 4, Compound 5 or Compound 6 with folpet, combinations of Compound 3, Compound 4, Compound 5 or Compound 6 with mancozeb, combinations of Compound 3, Compound 4, Compound 5 or Compound 6 with maneb, combinations of Compound 3, Compound 4, Compound 5 or Compound 6 with quinoxyfen, combinations of Compound 3, Compound 4, Compound 5 or Compound 6 with validamycin, combinations of Compound 3, Compound 4, Compound 5 or Compound 6 with vinclozolin, Compound 3, Compound 4, Compound 5 or Compound 6 with fenpropidine, combinations of Compound 3, Compound 4, Compound 5 or Compound 6 with fenpropimorph, combinations of Compound 3, Compound 4, Compound 5 or Compound 6 with bromuconazole, combinations of Compound 3, Compound 4, Compound 5 or Compound 6 with cyproconazole, combinations of Compound 3, Compound 4, Compound 5 or Compound 6 with difenoconazole, combinations of Compound 3, Compound 4, Compound 5 or Compound 6 with epoxyconazole, combinations of Compound 3, Compound 4, Compound 5 or Compound 6 with flusilazole, combinations of Compound 3, Compound 4, Compound 5 or Compound 6 with ipconazole, combinations of Compound 3, Compound 4, Compound 5 or Compound 6 with metconazole, combinations of Compound 3, Compound 4, Compound 5 or Compound 6 with propiconazole, combinations of Compound 3, Compound 4, Compound 5 or Compound 6 with tebuconazole, combinations of Compound 3, Compound 4, Compound 5 or Compound 6 with triticonazole, combinations of Compound 3, Compound 4, Compound 5 or Compound 6 with famoxadone, combinations of Compound 3, Compound 4, Compound 5 or Compound 6 with fenamidone, combinations of Compound 3, Compound 4, Compound 5 or Compound 6 with benomyl, combinations of Compound 3, Compound 4, Compound 5 or Compound 6 with cymoxanil, combinations of Compound 3, Compound 4, Compound 5 or Compound 6 with fosetyl-aluminum, combinations of Compound 3, Compound 4, Compound 5 or Compound 6 with metalaxyl, combinations of Compound 3, Compound 4, Compound 5 or Compound 6 with propineb, combinations of Compound 3, Compound 4, Compound 5 or Compound 6 with zineb, combinations of Compound 3, Compound 4, Compound 5 or Compound 6 with copper sulfate, combinations of Compound 3, Compound 4, Compound 5 or Compound 6 with copper hydroxide, combinations of Compound 3, Compound 4, Compound 5 or Compound 6 with propamocarb, combinations of Compound 3, Compound 4, Compound 5 or Compound 6 with cyazofamid, combinations of Compound 3, Compound 4, Compound 5 or Compound 6 with zoxamid, combinations of Compound 3, Compound 4, Compound 5 or Compound 6 with fluazinam and combinations of Compound 3, Compound 4, Compound 5 or Compound 6 with iprovalicarb. Compound numbers refer to compounds in Index Table A and B.

Formulation/Utility

Compositions of this invention will generally be used as a formulation or composition comprising at least one carrier selected from agriculturally suitable liquid diluents, solid diluents and surfactants. The formulation or composition ingredients are selected to be consistent with the physical properties of the active ingredient, mode of application and environmental factors such as soil type, moisture and temperature. Useful formulations include liquids such as solutions (including emulsifiable concentrates), suspensions, emulsions (including microemulsions and/or suspoemulsions) and the like which optionally can be thickened into gels. Useful formulations further include solids such as dusts, powders, granules, pellets, tablets, films, and the like which can be water-dispersible (“wettable”) or water-sbluble. Active ingredient can be (micro)encapsulated and further formed into a suspension or solid formulation; alternatively the entire formulation of active ingredient can be encapsulated (or “overcoated”). Encapsulation can control or delay release of the active ingredient. Sprayable formulations can be extended in suitable media and used at spray volumes from about one to several hundred liters per hectare. High-strength compositions are primarily used as intermediates for further formulation.

The formulations will typically contain effective amounts (e.g. from 0.01-99.99 weight percent) of active ingredients together with diluent and/or surfactant within the following approximate ranges which add up to 100 percent by weight.

| | Weight Percent | | |
|---|---|---|---|
| | Active | | |
| | Ingredients | Diluent | Surfactant |
| Water-Dispersible and Water- | 5-90 | 0-94 | 1-15 |
| soluble | | | |
| Granules, Tablets and | | | |
| Powders. | | | |
| Suspensions, Emulsions, | 5-50 | 40-95 | 0-25 |
| Solutions (including | | | |
| Emulsifiable | | | |
| Concentrates) | | | |
| Dusts | 1-25 | 70-99 | 0-5 |
| Granules and Pellets | 0.01-99 | 5-99.99 | 0-15 |
| High Strength Compositions | 90-99 | 0-10 | 0-2 |

Typical solid diluents are described in Watkins, et al., Handbook of Insecticide Dust Diluents and Carriers, 2nd Ed., Dorland Books, Caldwell, N.J. Typical liquid diluents are described in Marsden, Solvents Guide, 2nd Ed., lnterscience, New York, 1950. McCutcheon's Detergents and Emulsifers Annual, Allured Publ. Corp., Ridgewood, N.J., as well as Sisely and Wood, Encyclopedia of Surface Active Agents, Chemical Publ. Co., Inc., New York, 1964, list surfactants and recommended uses. All formulations can contain minor amounts of additives to reduce foam, caking, corrosion, microbiological growth and the like, or thickeners to increase viscosity.

Surfactants include, for example, polyethoxylated alcohols, polyethoxylated alkylphenols, polyethoxylated sorbitan fatty acid esters, dialkyl sulfosuccinates, alkyl sulfates, alkylbenzene sulfonates, organosilicones, N,N-dialkyltaurates, lignin sulfonates, naphthalene sulfonate formaldehyde condensates, polycarboxylates, and polyoxyethylene/polyoxypropylene block copolymers. Solid diluents include, for example, clays such as bentonite, montmorillonite, attapulgite and kaolin, starch, sugar, silica, talc, diatomaceous earth, urea, calcium carbonate, sodium carbonate and bicarbonate, and sodium sulfate. Liquid diluents include, for example, water, N,N-dimethylformamide, dimethyl sulfoxide, N-alkylpyrrolidone, ethylene glycol, polypropylene glycol, paraffins, alkylbenzenes, alkylnaphthalenes, oils of olive, castor, linseed, tung, sesame, corn, peanut, cotton-seed, soybean, rape-seed and coconut, fatty acid esters, ketones such as cyclohexanone, 2-heptanone, isophorone and 4-hydroxy-4-methyl-2-pentanone, and alcohols such as methanol, cyclohexanol, decanol and tetrahydrofurfuryl alcohol.

Solutions, including emulsifiable concentrates, can be prepared by simply mixing the ingredients. Dusts and powders can be prepared by blending and, usually, grinding as in a hammer mill or fluid-energy mill. Suspensions-are usually prepared by wet-milling; see, for example, U.S. Pat. No. 3,060,084. Preferred suspension concentrates include those containing, in addition to the active ingredient, from 5 to 20% nonionic surfactant (for example, polyethoxylated fatty alcohols) optionally combined with 50-65% liquid diluents and up to 5% anionic surfactants. Granules and pellets can be prepared by spraying the active material upon preformed granular carriers or by agglomeration techniques. See Browning, “Agglomeration”, Chemical Enginieering, Dec. 4, 1967, pp 147-48, Perry's Chemical Enginieer's Handbook, 4th Ed., McGraw-Hill, N.Y., 1963, pages 8-57 and following, and WO 91/13546. Pellets can be prepared as described in U.S. Pat. No. 4,172,714. Water-dispersible and water-soluble granules can be prepared as taught in U.S. Pat. No. 4,144,050, U.S. Pat. No. 3,920,442 and DE 3,246,493. Tablets can be prepared as taught in U.S. Pat. No. 5,180,587, U.S. Pat. No. 5,232,701 and U.S. Pat. No. 5,208,030. Films can be prepared as taught in GB 2,095,558 and U.S. Pat. No. 3,299,566.

For further information regarding the art of formulation, see U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; and Hance et al., Weed Control Handbook, 8th Ed., Blackwell Scientific Publications, Oxford, 1989.

In the following Examples, all percentages are by weight and all formulations are prepared in conventional ways. Without further elaboration, it is believed that one skilled in the art using the preceding description can utilize the present invention to its fililest extent. The folloing Examples are, therefore, to be construed as merely illustrative, and not limiting of the disclosure in any way whatsoever. Percentages are by weight except where otherwise indicated.

### EXAMPLE A

| Wettable Powder | |
|---|---|
| Active ingredients | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0%. |

### EXAMPLE B

| Granule | |
|---|---|
| Active ingredients | 10.0% |
| attapulgite granules (low volatile matter, | 90.0%. |
| 0.71/0.30 mm; U.S.S. No. 25-50 sieves) | |

### EXAMPLE C

| Extruded Pellet | |
|---|---|
| Active ingredients | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0%. |

### EXAMPLE D

| Emulsifiable Concentrate | |
|---|---|
| Active ingredients | 20.0% |
| blend of oil soluble sulfonates | 10.0% |
| and polyoxyethylene ethers | |
| isophorone | 70.0%. |

### EXAMPLE E

| Suspension Concentrate | | |
|---|---|---|
| Active ingredients | | 20.0% |
| polyethoxylated fatty alcohol | nonionic surfactant | 15.0% |
| ester derivative of montan wax | | 3.0% |
| calcium lignosulfonate | anionic surfactant | 2.0% |
| polyethoxylated/polypropoxylated | | |
| polyglycol block copolymer | surfactant | 1.0% |
| propylene glycol | diluent | 6.4% |
| poly(dimethylsiloxane) | antifoam agent | 0.6% |
| antimicrobial agent | | 0.1% |
| water | diluent | 51.9% |

The formulation ingredients are mixed together as a syrup, the active ingredients are added and the mixture is homogenized in a blender. The resulting slurry is then wet-milled to form a suspension concentrate.

Compositions of this invention can also be mixed with one or more insecticides, nematocides, bactericides, acaricides, growth regulators, chemosterilants, semiochemicals, repellents, attractants, pheromones, feeding stimulants or other biologically active compounds to form a multi-component pesticide giving an even broader spectrum of agricultural protection. Examples of such agricultural protectants with which compositions of this invention can be formulated are: insecticides such as abamectin, acephate, azinphos-methyl, bifenthrin, buprofezin, carbofuran, chlorfenapyr, chlolpyrifos, chlorpyrifos-methyl, cyfluthrin, beta-cyfluthrin, cyhalothrin, lambda-cyhalothrin, deltamethrin, diafenthiuron, diazinon, diflubenzuron, dimethoate, esfenvalerate, fenoxycarb, fenpropathrin, fenvalerate, fipronil, flucythrinate, tau-fluvalinate, fonophos, imidacloprid, isofenphos, malathion, metaldehyde, methamidophos, methidathion, methomyl methoprene, methoxychlor, methyl 7-chloro-2,5-dihydro-2-[[N-(methoxycarbonyl)-N-[4-(trifluoromethoxy)phenyl]amino]carbonyl]indeno[1,2-e][1,3,4]oxadiazine-4a(3H)-carboxylate(indoxacarb), monocrotophos, oxamyl, parathion, parathion-methyl, permethrin, phorate, phosalone, phosmet, phosphamidon, pirimicarb, profenofos, rotenone, sulprofos, tebufenozide, tefluthrin, terbufos, tetrachlorvinphos, thiodicarb, tralomethrin, trichlorfon and triflumuron; bactericides such as streptomycin; acaricides such as amitraz, chinomethionat, chlorobenzilate, cyhexatin, dicofol, dienochlor, etoxazole, fenazaquin, fenbutatin oxide, fenpropathrin, fenpyroximate, hexythiazox, propargite, pyridaben and tebufenpyrad; nematocides such as aldoxycarb and fenamiphos; and biological agents such as Bacillus thuringiensis, Bacillus thuiigiensis delta endotoxin, baculovirus, and entomopathogenic bacteria, virus and fungi. The weight ratios of these various mixing partners to compounds of Formula I of this invention typically are between 100:1 and 1:100, preferably between 30:1 and 1:30, more preferably between 10:1 and 1:10 and most preferably between 4:1 and 1:4.

The compounds and compositions of this invention are useful as plant disease control agents. The present invention therefore further comprises a method for controlling plant diseases caused by fungal plant pathogens comprising applying to the plant or portion thereof to be protected, or to the plant seed or seedling to be protected, an effective amount of a compound of the invention or a fungicidal composition containing said compound.

The preferred methods of use are those involving the compounds or compositions preferred above.

The compounds and compositions of this invention provide control of diseases caused by a broad spectrum of fungal plant pathogens in the Basidiomycete, Ascomycete, Oomycete and Deuteromycete classes. They are effective in controlling a broad spectrum of plant diseases, particularly foliar pathogens of ornamental, vegetable, field, cereal, and fruit crops. These pathogens include Plasmopara viticola, Phzytophthora infestans, Peronospora tabacina, Pseudoperonospora cubensis, Pythium aphanidermatum, Alternaria brassicae, Septora nodorum, Septoria tritici, Cercosporidium personatum, Cercospora arachidicola, Pseudocercosporella herpotrichoides, Cercospora beticola, Botrytis cinerea, Monilinia fructicola, Pyricularia oryzae, Podospharia leucotricha, Venturia inaequalis, Erysiphe graminis, Uncinula necatur, Puccinia reconidita, Puccinia graminis, Hemileia vastatrix, Puccinia striiformis, Puccinia arachidis, Rhizoctonia solani, Sphaerotheca fuliginiea, Fusarium oxysporum, Verticillium dahliae, Pythium aphanidermatum, Phytophthora megasperma, Sclerotinia sclerotiorum, Sclerotium rolfsii, Erysiphe polygoni, Pyrenophora teres, Gaeumannomyces graminis, Rynchosporium secalis, Fusaruim roseum, Bremia lactucae and other generea and species closely related to these pathogens. The compositions of the invention are especially effective in controlling Plasmopara viticola on grapes and Phltophthotia inifestaizs on potatoes and tomatoes.

Plant disease control is ordinarily accomplished by applying an effective amount of a compound of this invention either pre- or post-infection, to the portion of the plant to be protected such as the roots, stems, foliage, fruit, seeds, tubers or bulbs, or to the media (soil or sand) in which the plants to be protected are growing. The compounds can also be applied to the seed to protect the seed and seedling.

Rates of application for these compounds can be influenced by many factors of the environment and should be determined under actualuse conditions. Foliage can normally be protected when treated at a rate of from less than 1 g/ha to 5,000 g/ha of active ingredient. Seed and seedlings can normally be protected when seed is treated at a rate of from 0.1 to 10 g per kilogram of seed.

The following TESTS demonstrate the control efficacy of compounds of this invention on specific pathogens. The pathogen control protection afforded by the compounds is not limited, however, to these species. See Index Table A-C for compound descriptions. The abbreviation “Me” stands for “methyl” . The abbreviation “Ex.” stands for “Example” and is followed by a number indicating in which example the compound is prepared. The symbol “- -” means there is no substituent conresponding to that group.

INDEX TABLE A

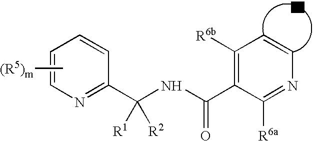

| Compound | | | | | | | | m.p. |
|---|---|---|---|---|---|---|---|---|
| Number | R¹ | R² | (R⁵)ₘ | R^{6a} | K | (R⁷)ₙ | R^{6b} | (° C.) |
| 1 | H | H | 3-Cl-5-CF₃ | Cl | K- | — | Cl | * |
| | | | | | 38 | | | |
| 2 | H | H | 3-Cl-5-CF₃ | Cl | K- | — | Cl | 158-160 |
| | | | | | 2 | | | |
| 3 (Ex. 1) | H | H | 3-Cl-5-CF₃ | Cl | K- | — | Cl | 48-50 |
| | | | | | 40 | | | |

INDEX TABLE B

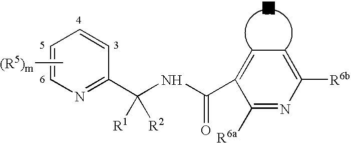

| Compound | | | | | | | | m.p. |
|---|---|---|---|---|---|---|---|---|
| Number | R¹ | R² | (R⁵⁾ₘ | R^{6a} | K | (R⁷)ₙ | R^{6b} | (° C.) |
| 4 | H | Me | 3-Cl-5-Br | Cl | K-38 | — | H | * |
| 5 | H | Me | 3-Cl-5-Br | Br | K-38 | — | H | * |
| 6 | H | Me | 3-Cl-5-Br | F | K-38 | — | H | * |

*See Index Table B for ¹H NMR data.

INDEX TABLE C

| | ¹H NMR Data(300 mHz; CDCl₃ |
|---|---|
| Cmpd No. | solution unless indicated otherwise) |
| 3 | δ 8.69(1H, s), 8.26(1H, d, J=8Hz), 8.09(1H, d, J=8Hz), |
| | 8.01(1H, s), 7.87(1H, t, J=8Hz), 7.73(1H, t, J=8Hz), |
| | 7.59(1H, br s), 5.04(2H, d, J=4Hz). |
| 4 | δ 9.09(s, 1H), 8.46(s, 1H), 8.00(d, 1H), 7.92(d, 1H), |
| | 7.89(d, 1H), 7.75(m, 1H), 7.64(m, 1H), 7.49(bd, 1H), |
| | 5.87(m, 1H), 1.66(d, 3H). |
| 5 | δ 9.05(s, 1H), 8.46(s, 1H), 8.00(d, 1H), 7.92(d, 1H), |
| | 7.87(d, 1H), 7.75(m, 1H), 7.66(m, 1H), 7.46(bd, 1H), |
| | 5.88(m, 1H), 1.67(d, 3H). |
| 6 | δ 9.01(s, 1H), 8.50(s, 1H), 8.28(d, 1H), 8.02(d, 1H), |
| | 7.92(d, 1H), 7.77(m, 1H), 7.66(bd, 1H), 7.62(m, 1H), |
| | 5.85(m, 1H), 1.63(d, 3H). |

### BIOLOGICAL EXAMPLES OF THE INVENTION

General protocol for preparing test suspensions: Test compounds are first dissolved in acetone in an amount equal to 3% of the final volume and then suspended at the desired concentration (in ppm) in acetone and purified water (50/50 mix) containing 250 ppm of the surfactant Trem® 014 (polyhydric alcohol esters). The resulting test suspensions are then used in the following tests. Spraying a 200 ppm test suspension to the point of run-off on the test plants is the equivalent of a rate of 500 g/ha.

### Test A

The test suspension was sprayed to the point of run-off on wheat seedlings. The following day the seedings are inoculated with a spore dust of Erysiphe graminis f. sp. tritici, (the causal agent of wheat powdery mildew) and incubated in a growth chamber at 20° C. for 7 days, after which disease ratings are made.

### Test B

The test suspension was sprayed to the point of run-off on wheat seedlings. The following day the seedlings are inoculated with a spore suspension of Puccinia recondita (the causal agent of wheat leaf rust) and incubated in a saturated atmosphere at 20° C. for 24 h, and then moved to a growth chamber at 20° C. for 6 days, after which disease ratings are made.

### Test C

The test suspension was sprayed to the point of run-off on potato seedlings. The following day the seedlings are inoculated with a spore suspension of Phytophthora infestans (the causal agent of tomato and potato late blight) and incubated in a saturated atmosphere at 20° C. for 24 h, and then Moved to a growth chamber at 20° C. for 5 days, after which disease ratings are made.

### Test D

The test suspension was sprayed to the point of run-off on tomato seedlings. The following day the seedlings are inoculated with a spore suspension of Phytophthora infestans (the causal agent of tomato and potato late blight) and incubated in a saturated atmosphere at 20° C. for 24 h, and then moved to a growth chamber at 20° C. for 5 days, after which disease ratings are made.

### Test E

The test suspension was sprayed to the point of run-off on grape seedlings. The following day the seedlings are inoculated with d spore suspension of Plasniopara viticola (the causal agent of grape downy mildew) and incubated in a saturated atmosphere at 20° C. for 24 h, moved to a growth chamber at 20° C. for 6 days, and then incubated in a saturated atmosphere at 20° C. for 24 h, after which disease ratings are made.

### Test F

Potato seedlings are inoculated with a spore suspension of Phytophthora infestans (the causal agent of potato and tomato late blight) and incubated in a saturated atmosphere at 20° C. for 24 h. The next day, test suspension is sprayed to the point of run-off and the treated plants are moved to a growth chamber at 20° C. for 5 days, after which disease ratings are made.

### Test G

Grape seedlings are inoculated with a spore suspension of Plaszopara viticola (the causal agent of grape downy mildew) and incubated in a saturated atmosphere at 20° C. for 24 h. The next day, test suspension is sprayed to the point of run-off and the treated plants are moved to a growth chamber at 20° C. for 6 days, and then incubated in a saturated atmosphere at 20° C. for 24 h, after which disease ratings are made.

Results for Tests A-G are given in Table A. In the table, a rating of 100 indicates 100% disease control and a rating of 0 indicates no disease control (relative to the controls). A dash (-) indicates no test results.

TABLE A

| Cmpd | Test | Test | Test | Test | Test | Test | Test |
|---|---|---|---|---|---|---|---|
| No. | A | B | C | t D | E | F | G |
| 1 | 0 | 0 | 98** | 97 | 99** | 0** | 15 |
| 2 | 0 | 0 | 100** | 100 | 100** | 93** | 13 |
| 3 | 0 | 0 | 100** | 100 | 100** | 0** | 100 |
| 4 | 0 | — | 100** | 100 | 100** | 0** | 100 |
| 5 | 0 | — | 100** | 100 | 100** | 85** | 92 |
| 6 | 0 | — | 100** | 100 | 100** | 24** | 96 |

**tested at 100 ppm

1. A compound selected from Formula I, and N-oxides and agriculturally suitable salts thereof, 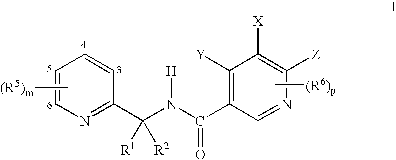
wherein
R¹ and R² are each independently H or C₁-C₆ alkyl;
X and either Y or Z are a linking chain 3 or 4 atoms in length attached to contiguous carbon atoms and are talcen together with said carbon atoms to form a fused phenyl ring, a fused 5- or 6-membered nonaromatic carbocyclic or heterocyclic ring optionally including one or two ring members selected from the group consisting of C(═O), SO and S(O)₂, or a fused 5- or 6-membered heteroaromatic ring, each fused ring optionally substituted with one to three substituents independently selected from R⁷;
each R⁵ is independently C₁-C₆ alkyl, C₂-C₆ alkenyl, C₂-C₆ alknyl, C₃-C₆ cycloalkyl, C₁-C₆ haloalkyl, C₂-C₆ haloalkenyl, C₂-C₆ haloalkynyl, C₃-C₆ halocycloalkyl, halogen, CN, CO₂H, CONH₂, NO₂, hydroxy, C₁-C₄ alkoxy, C₁-C₄ haloalkoxy, C₁-C₄ alkylthio, C₁-C₄ alkylsulfinyl, C₁-C₄ alkylsulfonyl, C₁-C₄ haloalkylthio, C₁-C₄ haloalkylsulfinyl, C₁-C₄ haloalkylsulfonyl, C₁-C₄ alkylamino, C₂-C₈ dialkylamino, C₃-C₆ cycloalkylamino, C₂-C₆ alkylcarbonyl, C₂-C₆ alkoxycarbonyl, C₂-C₆ alkylaminocarbonyl, C₃-C₈ dialkylaminocarbonyl or C₃-C₆ trialkylsilyl;
each R⁶ is independently C₁-C₆ alkyl, C₂-C₆ alkenyl, C₂-C₆ alkynyl, C₃-C₆ cycloalkyl, C₁-C₆ haloalkyl, C₂-C₆ haloalkenyl, C₂-C₆ haloalkynyl, C₃-C₆ halocycloalkyl, halogen, CN, CO₂H, CONH₂, NO₂, hydroxy, C₁-C₄ alkoxy, C₁-C₄ haloalkoxy, C₁-C₄ alklylthio, C₁-C₄ alkylsulfinyl, C₁-C₄ alkylsulfonyl, C₁-C₄ haloalkylthio, C₁-C₄ haloalkylsulfinyl C₁-C₄ haloalkylsulfonyl, C₁-C₄ alkylamino, C₂-C₈ dialkylamino, C₃-C₆ cycloalkylamino, C₂-C₆ alkylcarbonyl, C₂-C₆ alkoxycarbonyl, C₂-C₆ alkylaminocarbonyl, C₃-C₈ dialkylaminocarbonyl or C₃-C₆ trialkylsilyl;
each R⁷is independently C₁-C₄ alkyl, C₂-C₄ alkenyl, C₂-C₄ alkynyl, C₃-C₆ cycloalkyl, C₁-C₄ haloalkyl, C₂-C₄ haloalkenyl, C₂-C₄ haloalkynyl, C₃-C₆ halocycloalkyl, halogen, CN, NO₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ alkylamino, C₂-C₈ dialkylamino, C₃-C₆ cycloalkylamino, C₃-C₆ (alkyl)cycloalkylamino, C₂-C₄ alkylcarbonyl, C₂-C₆ alkoxycarbonyl, C₂-C₆ alkylaminocarbonyl, C₃-C₈ dialkylaminocarbonyl or C₃-C₆ triallcylsilyl;
m is 1, 2, 3 or 4; and
p is 0, 1, or 2.

2. The compound of claim 1 wherein X and either Y or Z and the carbon atoms to which they are attached form a fused phenyl ring, a fused 5- or 6-membered nonaromatic carbocyclic ring or a fused 5- or 6-membered nonaromatic heterocyclic ring, each fused ring optionally substituted with one to three substituents independently selected from R⁷.

3. The compound of claim 2 wherein one R⁵ is in the 3-position and a second R⁵ is in the 5-position and said two R⁵ groups are independently selected from the group consisting of C₁-C₆ alkyl, C₁-C₆ haloalkyl, halogen, CN, C₁-C₄ alkoxy, C₁-C₄ haloalkoxy C₁-C₄ alkylthio, C₁-C₄ alcylsulfinyl, C₁-C₄ alkylsulfonyl, C₁-C₄ haloalkylthio, C₁-C₄ haloalkylsulfinyl and C₁-C₄ haloalkylsulfonyl.

4. The compound of claim 3 wherein the R⁵ in the 3-position is selected from halogen and the R⁵ in the 5-position is selected firom the group. consisting of halogen, C₁-C₆ haloalkyl, C₁-C₄ haloalkoxy, C₁-C₄ haloalkylthio, C₁-C₄ haloalkylsulfinyl and C₁-C₄ haloalkylsulfonyl.

5. The compound of claim 4 wherein the R⁵ in the 3-position is selected from halogen and the R⁵ in the 5-position is selected from the group consisting of halogen, C₁-C₆ haloalkoxy and C₁-C₆ haloalkyl.

6. The compound of claim 5 wherein the R⁵ in the 3-position is chloro and the R⁵ in the 5-position is trifluoromethyl.

7. The compound of claim 5 wherein the R⁵ in the 3-position is chloro and the R⁵ in the 5-position is selected from halogen or C₁-C₆ haloalkoxy.

8. The compound of claim 1 wherein R¹ is H and R² is CH₃.

9. The compound of claim 1 wherein each R⁶ is independently selected from the group consisting of halogen, C₁-C₆ alkyl and C₁-C₆ haloalkyl.

10. The compound of claim 1 which is selected fiom the group:
2,4-dichloro-N[[3-chloro-5-(trifluoromethyl)-2-pyridinyl]methyl]-5,6,7,8-tetrahydro-3-quinolinecarboxamide;
N-[1 -5-bromo-3-chloro-2-pyndinyl)ethyl]3chloro-4isoquinolinecarboxamide; and
3-bromo-N-[1-(5-bromo-3-chloro-2-pyridinyl)ethyl]-4-isoquinolinecarboxamide; and
N-[1-(5-bromo-3-chloro-2-pyridinyl)ethyl]-3-fluoro-4-isoquinolinecarboxamide.

11. A fungicidal composition comprising a fungicidally effective amount of a compound of claim 1 and at least one additional component selected from the group consisting of agriculturally suitable surfactants, solid diluents and liquid diluents.

12. A fungicidal composition comprising a fungicidally effective combination mixture of at least one compound of claim 1 and at least one other fungicide.

13. The composition of claim 12 comprising (a) at least one compound of Formula I; and
(b) at least one compound selected fiom the group consisting of
(b1) alkylenebis(dithiocarbamate) fungicides;
(b2) compounds acting at the bc₁ complex of the fungal mitochondrial respiratory electron transfer site;
(b3) cymoxanil;
(b4) compounds acting at the demethylase enzyme of the sterol biosynthesis pathway;
(b5) morpholine and piperidine compounds that act on the sterol biosynthesis pathway;
(b6) phenylamide fungicides;
(b7) pyrimidinone fungicides;
(b8) phthalimides; and
(b9) fosetyl-aluminum.

14. The composition of claim 13 wherein the weight ratio of component (b) to component (a) is fiom 9:1 to 4.5:1.

15. A method for controlling plant diseases caused by fungal plant pathogens comprising applying to the plant or portion thereof, or to the plant seed or seedling, a fungicidally effective ambunt of a compound of claim 1 or a composition thereof.

16. A method for controlling plant diseases caused by fungal plant pathlogens comprising applying to the plant or portion thereof, or to the plant seed or seedling, a fungicidally effective amount of a composition of claim 11.

17. A method of making the compound of claim 10 consisting essentially of the procedure of Example 1.
